(19) United States
(12) Patent Application Publication
(10) Pub. No.: US 2013/0178609 A1
(43) Pub. Date: Jul. 11, 2013

(54) MODULAR CONSTRUCTION OF LIPOPHOSPHOLIPIDS

(71) Applicants: Jean-Pierre Haelters, Brest (FR); Mathieu Berchel, Brest (FR); Hélène Couthon-Gourvès, Bohars (FR); Paul-Alain Jaffrès, Bohars (FR)

(72) Inventors: Jean-Pierre Haelters, Brest (FR); Mathieu Berchel, Brest (FR); Hélène Couthon-Gourvès, Bohars (FR); Paul-Alain Jaffrès, Bohars (FR)

(73) Assignees: Centre National de la Recherche Scientifique - CNRS, Paris (FR); UNIVERSITE DE BRETAGNE OCCIDENTALE

(21) Appl. No.: 13/809,379

(22) PCT Filed: Jul. 11, 2011
(86) PCT No.: PCT/EP11/61788

§ 371 Date: Mar. 21, 2013

Related U.S. Application Data
(60) Provisional application No. 61/363,032, filed on Jul. 9, 2010.

(51) Int. Cl. C07F 9/6558; C07H 15/26

(52) U.S. Cl. CPC C07F 9/65586; C07F 9/65583; C07H 15/26
USPC 536/17.1; 548/119; 544/126

(57) ABSTRACT

The present invention relates to fluorescent and/or targeting lipophilic compounds having the general formula (R1O)(R2O)P(O)—R3-R4-R5 wherein R1 and R2 are each independently a linear or branched, saturated or unsaturated C2-C24 alkyl, or a linear or branched, saturated or unsaturated C2-C24 monoalkenyl or polyalkenyl, the polyalkenyl having from 2 to 4 double bonds, or a linear or branched, saturated or unsaturated C2-C24 monoalkinyl or polyalkinyl, the polyalkinyl having from 2 to 4 triple bonds; R3 is selected from 0, S, —C(R6)2-, —CH(R7)-, —C(S)—N(R6)-, —CH(SR7)-S—S— or —N(R6)- wherein R6 is a hydrogen atom or a C1-C4 alkyl, and R7 is a C1-C4 alkyl; R4 comprises at least one junction function and at least one linker; and R5 is at least one chemical fluorescent group or at least one targeting group. Methods for preparing said lipophilic compounds are also disclosed.

### BACKGROUND OF THE INVENTION

1. Field of the Invention

The present disclosure relates to the synthesis of fluorescent phospholipids, targeting phospholipids and targeting fluorescent phospholipids obtainable by a modular construction. The present disclosure relates in particular to fluorescent and/or targeting liphosphonates and lipophosphoramidates.

2. Background Art

Fluorescent microscopy is a technology which has been used over the last decades in molecular and cellular biology. This technology has been making use of fluorescent probes that can be employed alone or covalently bonded to a protein, a nucleic acid (e.g. DNA, SiRNA), or a disulfide derivative, with the aim of tracking these compounds in vitro or in vivo by microscopy. Fluorescent conjugates of lipids have been used for a panel of applications including, for instance, the evaluation of enzymatic activity (PLA2). In connection with the use of cationic lipids as carriers of nucleic acids, the incorporation of fluorescent lipids into a formulation is also a documented strategy to track liposomes or lipoplexes. The liposomes are supramolecular nano-objects that are commonly produced by the autoassembly of cationic lipids. The interaction of such kind of cationic liposomes with nucleic acids (e.g. pDNA) produces lipoplexes that constitute a promising class of therapeutics for inherited or acquired diseases. Nevertheless, the efficient delivery of pDNA or SiRNA ex vivo or in vivo still constitutes a challenging topic. Different classes of cationic lipids have been used for gene delivery since the pioneer works of Feigner and Behr.

Lipophosphonates and lipophosphoramidates have been proposed to carry pDNA into cells. This type of synthetic vectors has proved to be efficient, when formulated alone or concomitantly with a neutral helper lipid, for in vitro transfection but also for in vivo applications (transfection of lung—murine model).

### SUMMARY OF THE INVENTION

According to a first aspect, the present disclosure relates to a lipophilic compound having the following general formula (I):

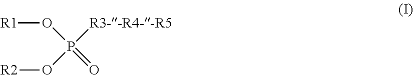

wherein:
(a) R1 and R2 are each independently a linear or branched, saturated or unsaturated C2-C24 alkyl, or a linear or branched, saturated or unsaturated C2-C24 monoalkenyl or polyalkenyl, the polyalkenyl having from 2 to 4 double bonds, or a linear or branched, saturated or unsaturated C2-C24 monoalkinyl or polyalkinyl, the polyalkinyl having from 2 to 4 triple bonds;
(b) R3 is selected from O, S, —C(R6)₂—, —CH(R7)-, —C(S)—N(R6)-, —CH(SR7)-S—S— or —N(R6)- wherein R6 is a hydrogen atom or a C1-C4 alkyl, and R7 is a C1-C4 alkyl;
(c) R4 comprises:
   (c1) at least one junction function selected from an ether group, a thioether group, an ester group, an amide group, a thioamide group, a carbonyl group, a carbamate group, an urea group, a thiourea group, a disulfide group and a 1,2,3-triazole; or
   (c2) at least one linker comprising a linear or branched, saturated or unsaturated hydrocarbon chain, the hydrocarbon chain being unsubstituted or substituted by one or a plurality of heteroatoms selected from N, O or S, interrupted and/or terminated by one or a plurality of junction functions selected from an ether group, a thioether group, an ester group, an amide group, a thioamide group, a carbonyl group, a carbamate group, an urea group, a thiourea group, a disulfide group and a 1,2,3-triazole, and optionally interrupted and/or terminated by one or a plurality of groups selected from C1-C4 alkyl radicals, C1-C4 alkoxy radicals and aryl radicals; and
(d) R5 is at least one chemical fluorescent group or at least one targeting group.

According to a second aspect, the present disclosure relates to methods for the preparation of lipophilic compounds according to the present invention. The methods comprise at least one coupling step between a Compound (A) and a Compound (B) by the formation of at least one covalent bond of the C-Het type or of the Het-Het′ type, wherein Het and Het′ are the same or different heteroatom(s), wherein Compound (A) is a compound of general formula (III):

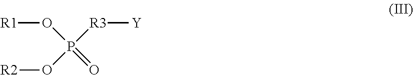

wherein:
   (g) R1, R2 and R3 are as described above;
   (h) Y is a grafting terminal functional group or unit selected from alcohol, phenol, thiol, amine, azide, dithioester, acylchloride, carboxylic acid, maleimide, isocyanates, isothiocyanate, alkyne, alkene, aziridine, epoxide, or vinyl;
wherein Compound (B) comprises R5 as described above and a grafting terminal functional group or unit Z selected from alcohol, phenol, thiol, amine, azide, dithioester, acylchloride, carboxylic acid, maleimide, isocyanates, isothiocyanate, alkyne, alkene, aziridine, epoxide, or vinyl and
wherein Z is capable of reacting with Y to form said covalent bond between Compound (A) and Compound (B).

In the present disclosure, R1 and R2 are also indicated by the terms “lipid part” and R5, when R5 is at least one fluorescent group, is also indicated by the terms “fluorophore” or “fluorescent group”.

### DETAILED DESCRIPTION OF EMBODIMENTS

To further investigate the cell trafficking of lipoplexes, the Applicant has performed their labelisation with fluorescent probes.

New phospholipids and the synthesis thereof are disclosed in the following.

The lipophilic compound according to a first aspect of the present invention has the following general formula (I):

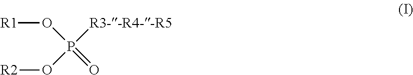

R1 and R2 are each a lipid chain. R1 and R2 may be identical or not, linear or branched, and may include no insaturations or one or several insaturations. The numbers of carbon atoms that are included in the lipid chains may be from 2 to 24. By modifying the structure of the lipid domain, different physico-chemical behaviors can be conferred to the lipophilic compound in terms, for example, of fluidity, fusogenicity, and in terms of modulation of the hydrophobic domain (such as size, volume).

According to a preferred embodiment of the present invention, R1 and R2 are each independently a linear or branched, saturated or unsaturated C2-C24 alkyl, or a linear or branched, saturated or unsaturated C2-C24 monoalkenyl or polyalkenyl, the polyalkenyl having from 2 to 4 double bonds, or a linear or branched, saturated or unsaturated C2-C24 monoalkinyl or polyalkinyl, the polyalkinyl having from 2 to 4 triple bonds.

According to a preferred embodiment of the present invention, R1 and R2 are each independently a linear or branched, saturated or unsaturated C10-C24 alkyl, or a linear or branched, saturated or unsaturated C10-C24 monoalkenyl or polyalkenyl, the polyalkenyl having from 2 to 4 double bonds, or a linear or branched, saturated or unsaturated C10-C24 monoalkinyl or polyalkinyl, the polyalkinyl having from 2 to 4 triple bonds.

According to another preferred embodiment, R1 and R2 are each independently a linear or branched, saturated or unsaturated C10-C24 alkyl, a linear or branched, saturated or unsaturated C10-C24 monoalkenyl or a linear or branched, saturated or unsaturated C10-C24 monoalkinyl.

According to a preferred embodiment, R1 is selected from a linear or branched, saturated or unsaturated C2-C4 alkyl, or a linear or branched, saturated or unsaturated C2-C4 monoalkenyl or polyalkenyl, the polyalkenyl having from 2 to 3 double bonds, or a linear or branched, saturated or unsaturated C2-C4 monoalkinyl or polyalkinyl, and R2 is selected from a linear or branched, saturated or unsaturated C14-C24 alkyl, or a linear or branched, saturated or unsaturated C14-C24 monoalkenyl or polyalkenyl, the polyalkenyl having from 2 to 4 double bonds, or a linear or branched, saturated or unsaturated C14-C24 monoalkinyl or polyalkinyl, the polyalkinyl having from 2 to 4 triple bonds.

According to another preferred embodiment, R1 is selected from a linear or branched, saturated or unsaturated C2-C4 alkyl, a linear or branched, saturated or unsaturated C2-C4 monoalkenyl or a linear or branched, saturated or unsaturated C2-C4 monoalkinyl, and R2 is selected from a linear or branched, saturated or unsaturated C14-C24 alkyl, a linear or branched, saturated or unsaturated C14-C24 monoalkenyl or a linear or branched, saturated or unsaturated C14-C24 monoalkinyl.

In a more preferred embodiment, R1 and R2 are each independently selected from the lipid chains shown below in Figure 1.

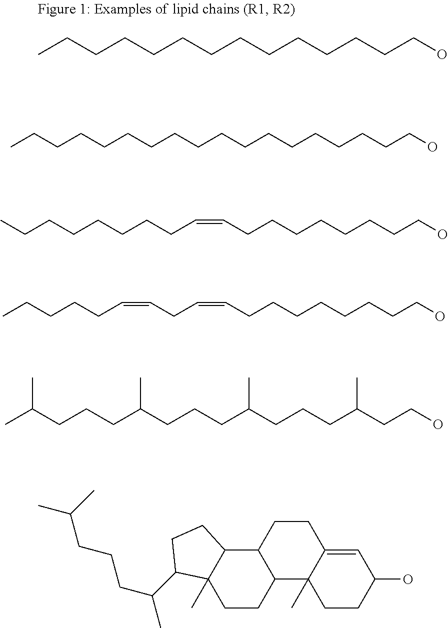 [SMILES: CCCCCCCCCCCCCCO ; CCCCCCCCCCCCCCCCCCO ; CCCCCCCC/C=C\CCCCCCCCO ; CCCCC/C=C\C/C=C\CCCCCCCCO ; CC(C)CCCC(C)CCCC(C)CCCC(C)CCO ; CC(C)CCCC(C)C1CCC2C3CCC4=CC(O)CCC4(C)C3CCC12C]

R3 is selected from O, S, —C(R6)₂—, —CH(R7)-, —C(S)—N(R6)-, —CH(SR7)-S—S— or —N(R6)- wherein R6 is a hydrogen atom or a C1-C4 alkyl, and R7 is a C1-C4 alkyl. In a preferred embodiment, R3 is —N(H)—.

R4 comprises at least one junction function or at least one linker.

The junction function is a functional group that allows the covalent junction between the phopholipid part and the fluorescent group and/or the targeting group.

Several reactions can be used to form the junction functions as reported below, for example triazole functional groups synthesized by <<click chemistry>> (reported for example in Sharpless et al., Angewandte Chemie International Edition 2001, 40, 2004-2021, A. J. Dirk et al, Chem. Comm., 2005, 4172-4174, R. K. Iha, Chem. Rev, 2009, 109, 5620-5686), amides or ester functional groups, thiol-ene and thiol-yne click chemistry (reported for example in Macromolecules, 2010, 43, 4113-4119 JACS, 2009, 131, 14673-14675, JACS, 2008, 130, 5062; JOC, 2008, 73, 3646; Chem. Pharm. Bull. 1975, 23, 2415; Chem. Phys. Lipids, 2000, 105, 215), thioamide (Phosphorus Sulf. Silicon, 1994, 89, 119-132), phosphorylated dithioacetal disulfide (Bulpin et al., J. Org. Chem., 1992, 57, 4507-4512), urea and thiourea (Diaz-Moscoso A., Chem. Eur. J., 2009, 15, 12871-12888).

Triazole Functional Group Synthesized by <<Click Chemistry>>

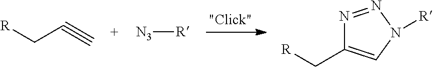

Amide or Ester Functional Group

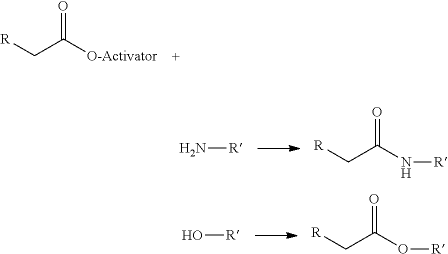

Thiol-ene and Thiol-yne Click Chemistry

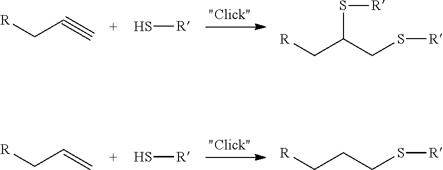

Thioamide

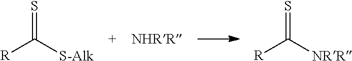

Phosphorylated Dithioacetal Disulfide

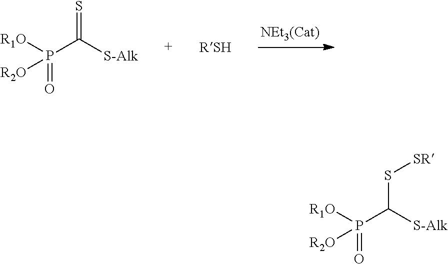

Urea and Thiourea

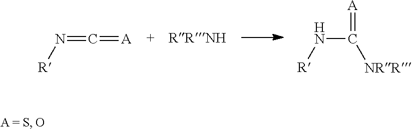

According to a preferred embodiment of the present invention, the junction function is selected from an ether group, a thioether group, an ester group, an amide group, a thioamide group, a carbonyl group, a carbamate group, an urea group, a thiourea group, a disulfide group and a 1,2,3-triazole.

In a preferred embodiment, the junction function is selected from an amide group, a 1,2,3-triazole, a thioether group, a thioamide group, an urea group or a thiourea group.

According to an embodiment of the present invention, the linker comprises a linear or branched, saturated or unsaturated hydrocarbon chain, the hydrocarbon chain being unsubstituted or substituted by one or a plurality of heteroatoms selected from N, O or S, interrupted and/or terminated by one or a plurality of junction functions selected from an ether group, a thioether group, an ester group, an amide group, a thioamide group, a carbonyl group, a carbamate group, an urea group, a thiourea group, a disulfide group and a 1,2,3-triazole, and optionally interrupted and/or terminated by one or a plurality of groups selected from C1-C4 alkyl radicals, C1-C4 alkoxy radicals and aryl radicals.

In a preferred embodiment, the linker may comprise an alkyl chain, an aryl chain or a polyethyleneglycol (PEG) chain. A combination of these structures is also possible.

In an embodiment, the linker comprises a PEG moiety, for example a sequence of linear ethylene glycol units, which may be of adjustable length:

O—CH₂—CH₂ₙ

In an embodiment, n in the PEG moiety is comprised between 1 and 150. In a preferred embodiment, n is comprised between 1 and 60. In a more preferred embodiment, n is comprised between 1 and 40. In a more preferred embodiment, the PEG moiety is a tetraethyleneglycol moiety.

It has been found that the incorporation of PEGylated lipid into liposomes or lipoplexes can modify both the interaction of the nano-object with the cell membranes and the systemic circulation time. The modulation of the length of the PEG moiety has been considered as an adjustable parameter in the synthesis scheme, thus making possible to tailor the physico-chemical behaviours of these nano-objects.

In an embodiment, the linker comprises an aryl structure, which may be substituted at one or more positions.

In an embodiment, the linker comprises an alkyl structure, i.e., —(CH₂)ₘ—, wherein m is comprised between 1 and 18, and more preferably between 1 and 10.

Thanks to the linker, the length between the fluorescent probe and the phospholipid functional group can be tuned.

R5 is at least one chemical fluorescent group or at least one targeting group.

According to an embodiment, R5 may be a chemical fluorescent group selected from organic fluorophores.

Different fluorophores can be included in the molecular construction. Several examples are depicted below in Table 1 with the indication of the functional group used to include these chromophores into the construction.

TABLE 1

| Name | Structure | References |
|---|---|---|
| 5-(6) Carboxyrhodamine | 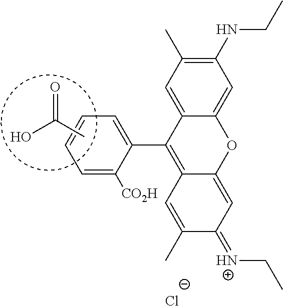 | Su-Chun Hung et al, Analytical biochem. 1996, 238, 165-170 |
| 5-(6) Carboxyfluorescein | 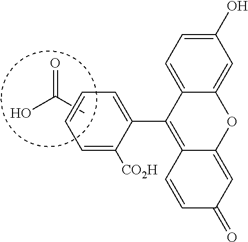 | Christopher R. Parish. Immunology and Cell Biology 1999, 77, 499- 508 |
| NBD | 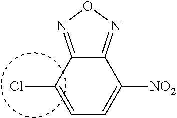 [SMILES: O=[N+]([O-])c1ccc(Cl)c2nonc12] | Robert Bittman et al. Biophysical Journal 2006, 90, 2170- 2178 Yasuhiko Koezuka et al. Org. Lett. 1999, 1, 359-361. |
| Coumarin | 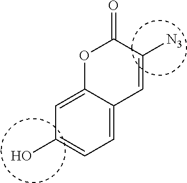 [SMILES: [N-]=[N+]=Nc1cc2ccc(O)cc2oc1=O] | Jeroen J. L. M. Cornelissen et al, Bioconjugate Chem. 2009, 20, 1129-1138. Qian Wang et al, Org. Lett. 2004, 6, 4603-4606. Christoph J. Fahrni, J. Am. Chem. Soc. 2004, 126, 8862-8863. Robert Bittman et al J. Org. Chem 2009, 74, 8844-8847. |
| 1,8-naphthalic anhydride | 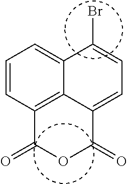 [SMILES: O=C1OC(=O)c2ccc(Br)c3cccc1c23] | Torfinnur Gunnlaugsson et al, Tetrahedron Lett. 2003, 44, 6575-6578. Chi-Huey Wong et al, proc. natl. acad. sci. Usa 2006, 103,12371-12376. |
| Anthracene | 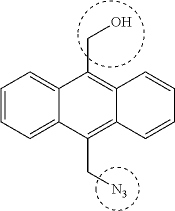 | Qian Wang et al, Tetrahedron 2008, 64, 2906- 2914. |
| BODIPY | 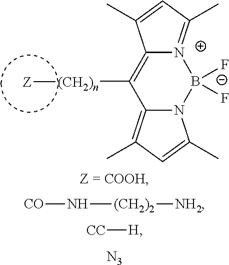 | Li et al., J. Org. Chem., 2006, 71, 1718-1721 Robert Bittman et al. Histochem Cell Biol 2008, 130, 819-832 Anal. Biochem. 378 (2008) 166-170 Bioorg. Med. Chem. Lett. 17 (2007) 6169-6171 Tetrahedron Letters 50 (2009) 6442-6445 |
| Cyanine | 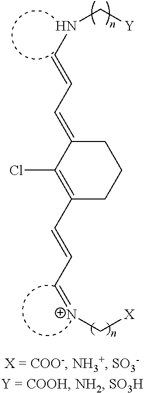 | Narayaman, N. ; Patonay, G. J. Org. Chem. 1995, 60, 2391-2395. Narayanan, N.; Strekowski, L.; Lipowska, M. ; Patonay, G. J. Org. Chem. 1997, 62, 9387-9387. Peng, X.; Song, F.; Lu, E.; Wang, Y.; Zhou, W.; Fan, J.; Gao, Y. J. Am. Chem. Soc. 2005, 727, 4170-4171. Samanta, A.; Vendrell, M.; Das, R.; Chang, Y.-T. Chem. Commun. 2010, 46, 7406-7408. |

In a preferred embodiment, the fluorescent group comprises an organic fluorophore, which can be for example selected from NBD, rhodamine, fluorescein, coumarin, anthracene, cyanine.

In a preferred embodiment, R5 is selected from couramin, 1,8-naphthalimide, anthracene and cyanine derivatives.

In a preferred embodiment, the at least one chemical fluorescent group is selected from—fluorescein and derivates thereof, near infrared absorbing and emitting fluorescent dyes, cyanine dyes, 7-hydroxy-9H-(1,3-dichloro-9,9-dimethylacridin-2-one), rhodamine and derivates thereof, amine-reactive fluorescent dyes, fluorescent dyes sold under the trade names BODIPY (R), IRDye (R) 800, Alexa Fluor (R) 750 and Alexa Fluor (R) 633, porphyrines, cyanines, oxazines and fluorescent nanoparticles.

R5 may be at least one targeting group. In a preferred embodiment, the targeting group may be selected from saccharides, disaccharides, and polysaccharides, such as for example mannose, galactose, lactose, anisamides and derivatives for sigma receptor targeting, peptides, such as for example RGD for integrin targeting, folate, antibody, phosphonic and di-phosphonic acids, and polyethyleneglycol (PEG) chain which may be of adjustable length. In a preferred embodiment, R5 is a polyethyleneglycol (PEG) chain which allows for the targeting of tumor tissues through Enhanced Permeability and Retention (EPR) effect.

The selective delivery of nano-objects may be based on the use of targeting moiety, such as for example saccharides, peptides, antibodies or a polyethyleneglycol (PEG) chain (EPR effect), which can be placed at the external side of the nano-objects. One way to visualize the trafficking of this kind of nano-objects in vitro or in vivo comprises the incorporation of a fluorescent lipid or probe into the formulation. Without being bound to a theory, it is believed that the fluorescent label and the targeting group are travelling together despite the absence of covalent link.

According to a preferred embodiment of the present disclosure, when R5 is at least one chemical fluorescent group, the lipophilic compound has the following general formula (II):

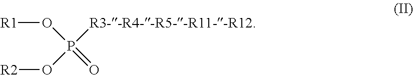

R1, R2, R3 and R4 are as described above. R5 is at least one chemical fluorescent group as described above. R11 comprises at least one junction function or at least one linker as described above in reference to R4. R12 is at least one targeting group as described above in reference to R5.

In a preferred embodiment of the present invention, when R4 comprises a linker, said linker comprises a chain terminated by two junction functions, a first junction function being covalently bonded to R3 and a second junction function being covalently bonded to R5.

In a preferred embodiment of the present invention, when R11 comprises a linker, said linker comprises a chain terminated by two junction functions, a first junction function being covalently bonded to R5 and a second junction function being covalently bonded to R12.

According to a preferred embodiment, the lipohilic compound of the present invention is selected from:

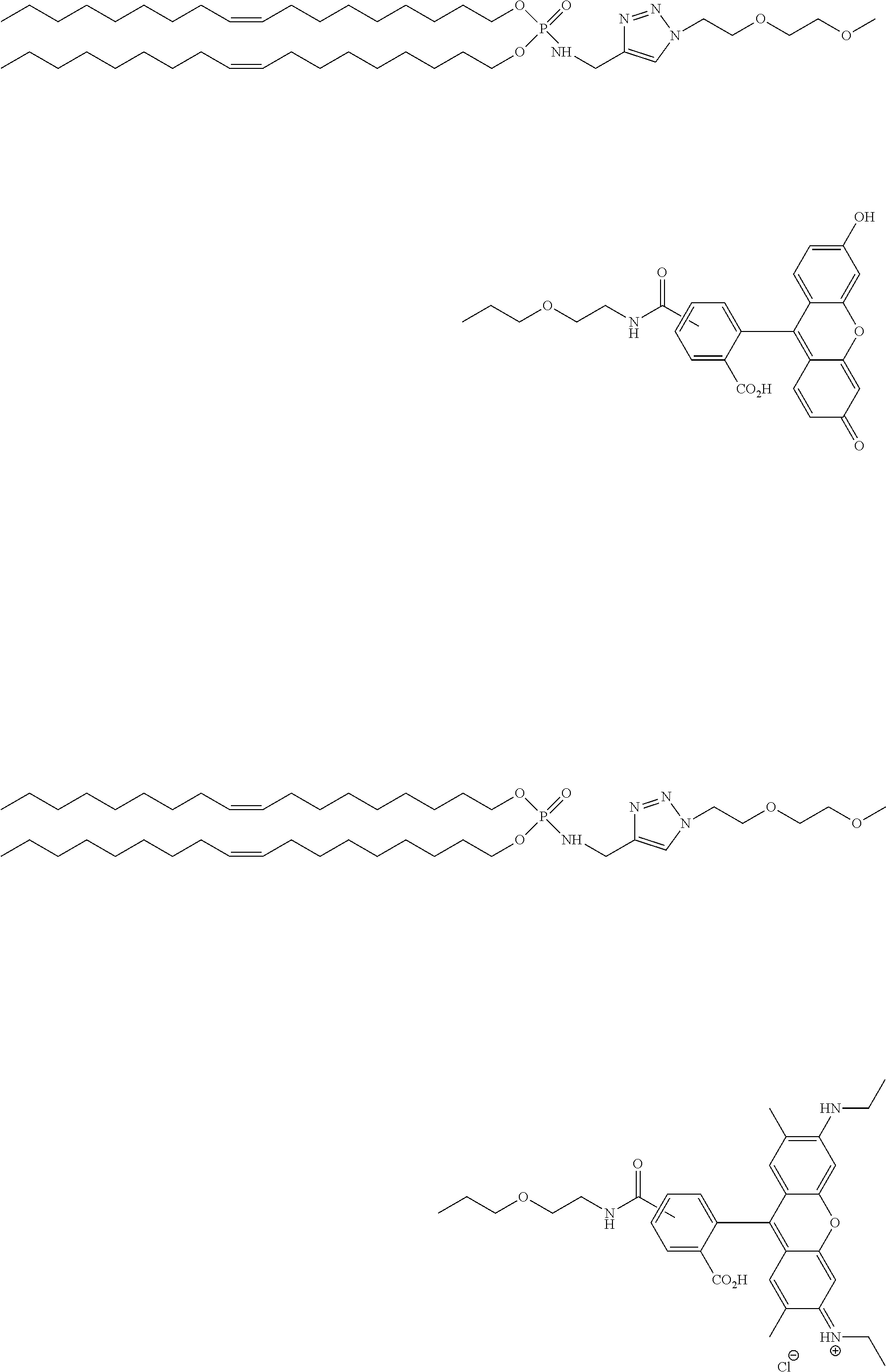

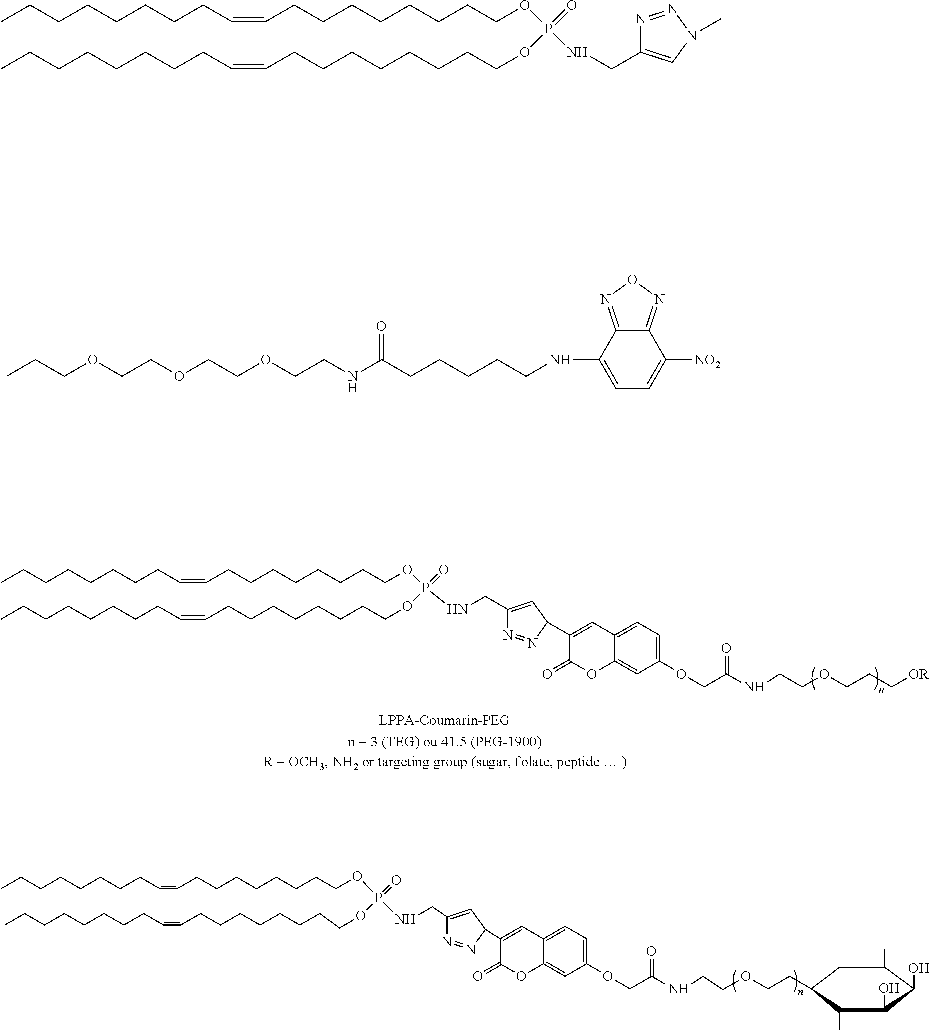

According to a further aspect of the present invention, the present disclosure relates to liposomes comprising the lipohilic compound according to the present invention.

According to yet another aspect of the present invention, the present disclosure relates to the use of the lipohilic compound according to the present invention for the preparation of a liposome.

The synthesis of the phospholipids of the present invention can be performed according to a modular convergent way.

Since the synthesis of lipophosphonates and lipophosphoramidates allows the introduction of a wide variety of chemical functionalities, a fluorescent tag and/or a targeting group have been incorporated into their structure. In the following, the synthesis of lipophosphonates and lipophosphoramidates comprising one fluorophore and/or one targeting group in the polar part is disclosed.

A possible general scheme associated to the fluorescent phospholipids according to an embodiment of the present invention is shown in Figure 2.

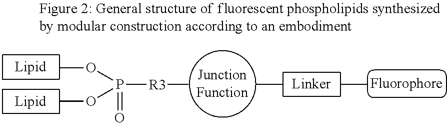

According to Figure 2, the fluorescent phospholipids of the present invention comprise three main parts: a lipid part, a linker and a fluorophore as described above. These three main parts can be joined together by at least one junction functional group as described above. The junction functional groups can be identical to or different from each other.

According to an embodiment, the phospholipids of the present invention may not comprise any linker as depicted in Figure 3 below.

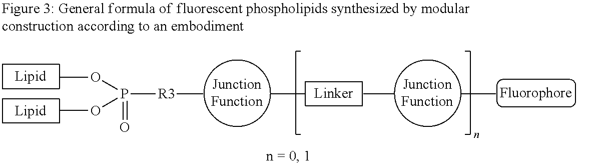

In an embodiment, the fluorophore can be separated from the lipid part by more than one linker as shown for example in Figure 4 below.

The following Figure 4 shows another embodiment of modular construction of the fluorescent phospholipids according to the present invention.

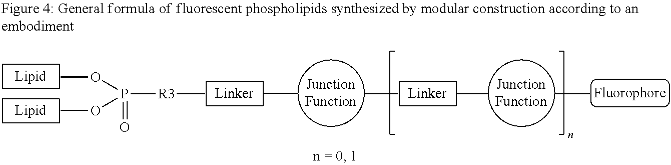

In targeting phospholipids, the modular way of construction allows to simplify the molecular construction for example by placing only the lipid part and the targeting group. According to an embodiment, the general formula is depicted in Figure 5. Possible structures of the lipid part, the linker, the junction function and the targeting group have been defined above.

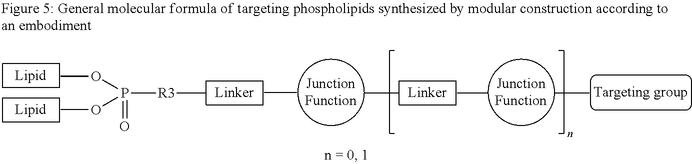

According to a second aspect, the present disclosure relates to methods for the preparation of lipophilic compounds according to the present invention. The methods comprise at least one coupling step between a Compound (A) and a Compound (B) by the formation of at least one covalent bond of the C-Het type or of the Het-Het′ type, wherein Het and Het′ are the same or different heteroatom(s), wherein Compound (A) is a compound of general formula (III):

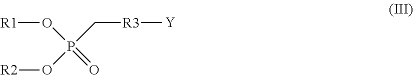

wherein:
   (g) R1, R2 and R3 are as described above;
   (h) Y is a grafting terminal functional group or unit selected from alcohol, phenol, thiol, amine, azide, dithioester, acylchloride, carboxylic acid, maleimide, isocyanates, isothiocyanate, alkyne, alkene, aziridine, epoxide, or vinyl;
wherein Compound (B) comprises R5 as described above and a grafting terminal functional group or unit Z selected from alcohol, phenol, thiol, amine, azide, dithioester, acylchloride, carboxylic acid, maleimide, isocyanates, isothiocyanate, alkyne, alkene, aziridine, epoxide, or vinyl; and
wherein Z is capable of reacting with Y to form said covalent bond between Compound (A) and Compound (B).

According to a preferred embodiment, Compound (A) and Compound (B) are coupled through a Compound (C) by the formation of at least one covalent bond of the C-Het type or of the Het-Het′ type, wherein Het and Het′ are the same or different heteroatom(s), wherein Compound (C) comprises R4 as described above and two grafting terminal functional groups or units W and X selected each independently from alcohol, phenol, thiol, amine, azide, dithioester, acylchloride, carboxylic acid, maleimide, isocyanates, isothiocyanate, alkyne, alkene, aziridine, epoxide, or vinyl. In such an embodiment, Z is capable of reacting with X to form a covalent bond between Compound (B) and Compound (C) and Y is capable of reacting with W to form a covalent bond between Compound (A) and Compound (C).

According to a further embodiment, the method further comprises at least one further coupling step between Compound (B) and a Compound (D) by the formation of at least one covalent bond of the C-Het type or of the Het-Het′ type, wherein Het and Het′ are the same or different heteroatom(s). In such an embodiment, R5 of Compound (B) is a chemical fluorescent group, Compound (B) further comprises a grafting terminal functional group or unit U selected from alcohol, phenol, thiol, amine, azide, dithioester, acylchloride, carboxylic acid, maleimide, isocyanates, isothiocyanate, alkyne, alkene, aziridine, epoxide, or vinyl; and Compound (D) comprises R12 as described above and a grafting terminal functional group or unit M selected from alcohol, phenol, thiol, amine, azide, dithioester, acylchloride, carboxylic acid, maleimide, isocyanates, isothiocyanate, alkyne, alkene, aziridine, epoxide, or vinyl. M is capable of reacting with U to form said covalent bond between Compound (B) and Compound (D).

According to a preferred embodiment, Compound (B) and Compound (D) are coupled through a Compound (E) by the formation of at least one covalent bond of the C-Het type or of the Het-Het′ type, wherein Het and Het′ are the same or different heteroatom(s), wherein Compound (E) comprises R11 as described above and two grafting terminal functional groups or units T and Q selected each independently from alcohol, phenol, thiol, amine, azide, dithioester, acylchloride, carboxylic acid, maleimide, isocyanates, isothiocyanate, alkyne, alkene, aziridine, epoxide, or vinyl. In such an embodiment, M is capable of reacting with Q to form a covalent bond between Compound (D) and Compound (E) and T is capable of reacting with U to form a covalent bond between Compound (B) and Compound (E).

The synthesis of targeting fluorescent phospholipids according to a modular way of construction is disclosed herein. The modular construction can be depicted by the general formula presented in Figure 6.

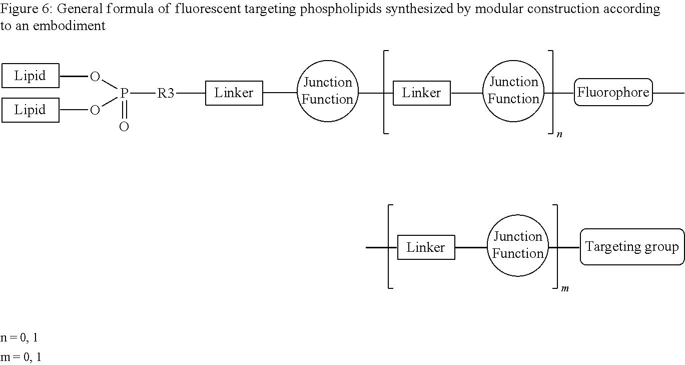

Examples of phospholipid building units, i.e. possible intermediates of the phospholipids of the present invention, used in the synthesis of the phospholipids of the present invention comprising a linker are shown in Figure 7 below.

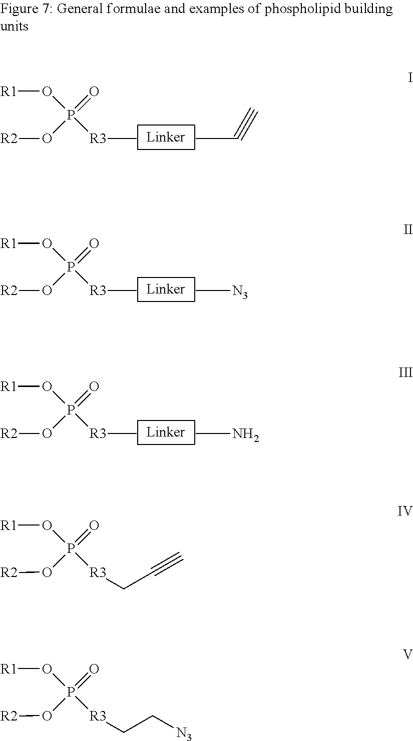

In Figure 7, compounds IV and V are two structures in which the structure of the linker is respectively methylene (CH₂) and ethylene (CH₂—CH₂).

Further examples of phospholipid building units used in the synthesis of the phospholipids of the present invention when they do not comprise a linker are shown in Figure 8 below.

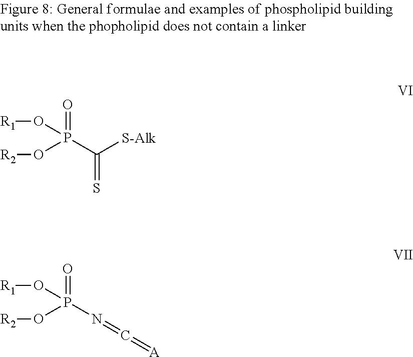

In an embodiment, a permanent or acquired cationic charge can incorporated in the molecular construction to produce cationic fluorescent phospholipids, as shown for example in Figure 9 below.

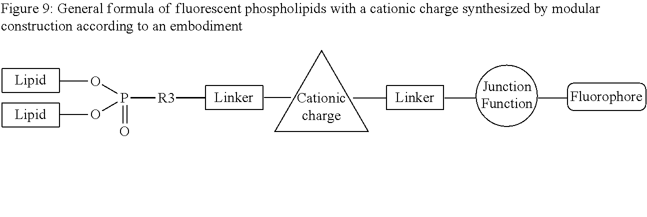

In order to design fluorescent targeting phospholipids, the building units reported (Figures 10 and 11) below can be used for the introduction of the targeting moiety before their formulation as liposomes or after their incorporation into a liposomal formulation.

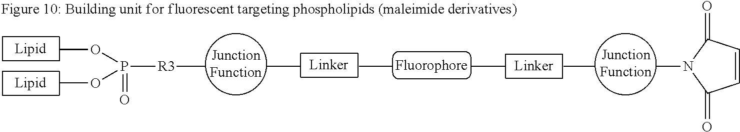

References are for example R. Koole et al, Bioconjugate chem. 2008, 19, 2471-2479, J. T. Ellioitt et al., Bioconjugate Chem, 2000, 11, 832-841, Kim, Eui-Sook; Son, Seong-Kil; Lee, In-Ho; Yoo, In-Kee; Kang, Sang-Jin, PCT Int. Appl. (2008), WO 2008156327 A2 20081224, Beduneau, Arnaud; Saulnier, Patrick; Hindre, Francois; Clavreul, Anne; Leroux, Jean-Christophe; Benoit, Jean-Pierre, Biomaterials, 2007, 28, 4978-4990.

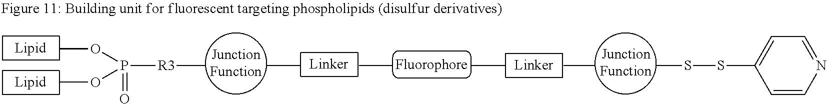

References are for example C. Chaix et al., Coll. Surf. B, Biointerf., 2003, 29, 39-521, Bettinger et alm., Bioconjugate Chem., 1998, 9, 842-846, Mercadal M; Domingo J C; Petriz J; Garcia J; de Madariaga M A Biochimica et biophysica acta 2000, 1509, 299-310.

For the synthesis of the fluorescent and/or targeting phospholipids of the present disclosure, a convergent method ended by a Huisgen “click” reaction is disclosed. However, other synthesis methods can be envisaged. The fluorescent and/or targeting phospholipids produced can be used to track for example lipoplexes, polyplexes, lipoplolyplexes and, in principle, any kind of lipid vesicles.

The Atherton-Todd reaction (reaction of a dialkylphosphite with an amine) is a method of choice for the synthesis of phosphoramidates. The use, in this reaction, of dialkylphosphite possessing two long alkyl chains (synthesized from diphenylphosphite and alkyl alcohol) has been extensively employed for the production of amphiphiles that were studied as gene carriers. The use, in this Atherthon-Todd reaction, of an alkyl amine functionalised by a terminal alkyne or azide functional group represents a quite direct synthesis pathway to produce lipid building blocks that can be “clicked” by making use of the Huisgen reaction to produce lipid-conjugates. According to this synthesis strategy, alkyne and azide lipo-phosphoramidates have been first synthesized and then have been employed via a Huisgen click coupling to produce fluorescent lipids.

The involvement of propargylamine in a coupling such that the Atherton-Todd reaction to produce lipophosphoramidates is disclosed herein to produce a molecular building block for a modular construction of phospholipids according to the present invention.

As depicted in Scheme 1 below, the O,O-dioleyl-N-propargylphosphoramidate (Compound 2) is obtained in a yield of 85% by using dioleylphosphite (Compound 1) and propargylamine as substrates.

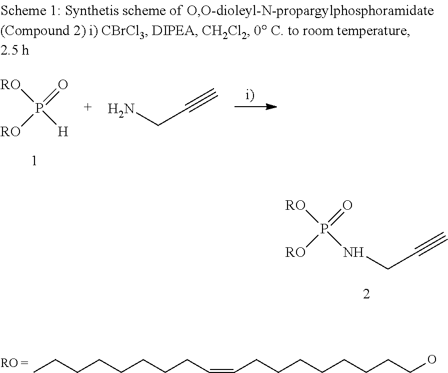

Compound 2, which can be obtained at a multi-gram scale, has a structure suitable for the design of lipoconjugates by making use of the Huisgen click reaction. To this purpose, Compound 2 was first engaged in a click reaction that uses the 3-azido-7-hydroxycoumarin (Compounds 3a and 3b) acting as the fluorescent building block (Scheme 2). The coumarin-lipophosphoramidates (Compounds 4a and 4B) were synthesized in 91% and 72% yield respectively.

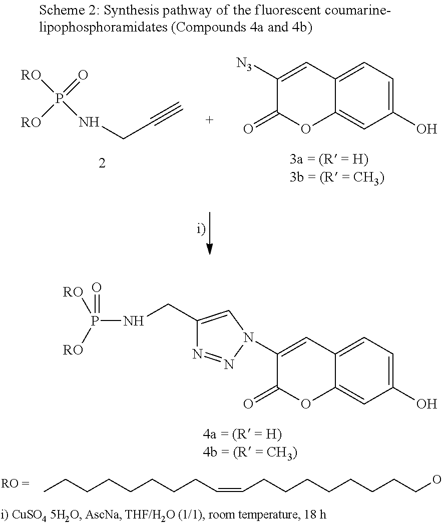

In Compounds 4a and 4b, the lipid part, that can act as a hydrophobic anchor, was separated from the fluorescent group by only a methylene unit. To tune the physico-chemical behavior of lipoplexes formulation, the length and the hydrophilic character of the linker that separates the lipid part from the fluorescent probe can be adjusted. Accordingly, the introduction of PEG moieties as linkers has been investigated. For this purpose different strategies can be use. In a first approach, the synthesis of the analogue (Compound 7) of Compound 2 in which the methylene unit is replaced by a tetraethyleneglycol (TEG) moiety has been achieved. The synthesis can start from tetraethyleneglycol (TEG) that can be mono alkylated with propargylbromide to produce Compound 5 (Scheme 3). This was done according to a procedure disclosed in Z.-B. Li, Z. Wu, K. Chen, F. T. Chin, and X. Chen Bioconjugate Chem. 2007, 18, 1987-1994, and in O. Norberg, L. Deng, M. Yan, and O. Ramstrom Bioconjugate Chem. 2009, 20, 2364-2370, which are both herein incorporated by reference.

Then the alcohol functional group was transformed in primary amine to give an amino-tetraethyleneglycol-alkyne (Compound 6). This can be done by any available procedure (such as for example mesylation, nucleophilic substitution with sodium azide and azide reduction). Compound 6 was then engaged in an Atherton-Todd reaction with O,O-dioleylphosphite to produce the lipophosphoramidate-PEG-alkyne (Compound 7) (yield: 72%), which can be easily functionalized by click reaction.

Compound 7 has been engaged in a click reaction with the 3-azido-6-hydroxycoumarin (Compound 3a) to obtain lipido-PEG conjugate of 6-hydroxycoumarin (Compound 8) (yield: 80%).

Compounds 4 and 8 have the same fluorescent properties, indicating that the linker (methylene or tetraethylene glycol) has no effect on the fluorescent properties of the coumarin probe.

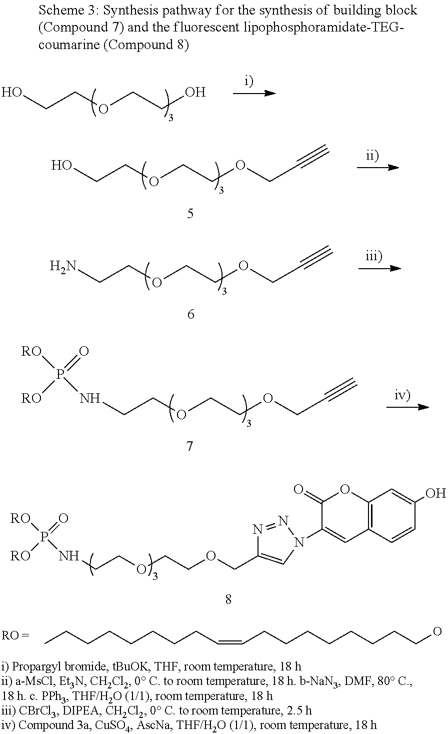

Different fluorescent lipids comprising different fluorescent probes, thus having different absorption/emission wave lengths, were obtained, thus rendering these compounds applicable to the field of molecular biology. In an embodiment, fluorescein was first used. For the conjugation of fluorescein to a lipophosphoramidate motif via click chemistry, fluorescein itself was used as starting material.

Two synthesis pathways have been performed that are differentiated by the sense of the click reaction which in an embodiment can involve one azide-lipophosphoramidate and one alkyne-fluorescein, and, in another embodiment, one alkyne-lipophosphoramidate and one azide-fluorescein.

According to a first pathway (Scheme 4), the NHS activated carboxylic acid of fluorescein reacts with ω-amino-azide-teraethyleneglycol (Compound 12) (prepared from tetraethylene glycol according to the procedure reported in C. R. Bertozzi and M. D. Bednarski J. Org. Chem. 1991, 56 4326-4329; b) S. S. Iyer, A. S. Anderson, S. Reed, B. Swanson and J. G. Schmidt Tetrahedron Lett. 2004, 45, 4285-4288, which is incorporated herein by reference—Scheme 5) to produce the clickable azido-fluorescein (Compound 9) in a yield of 85%. This compound was then engaged in the Huisgen reaction with the propargyl-lipophosphoramidate (Compound 2) to produce the desired lipophosphoramidate-TEG-fluorescein (Compound 10) in a yield of 84%.

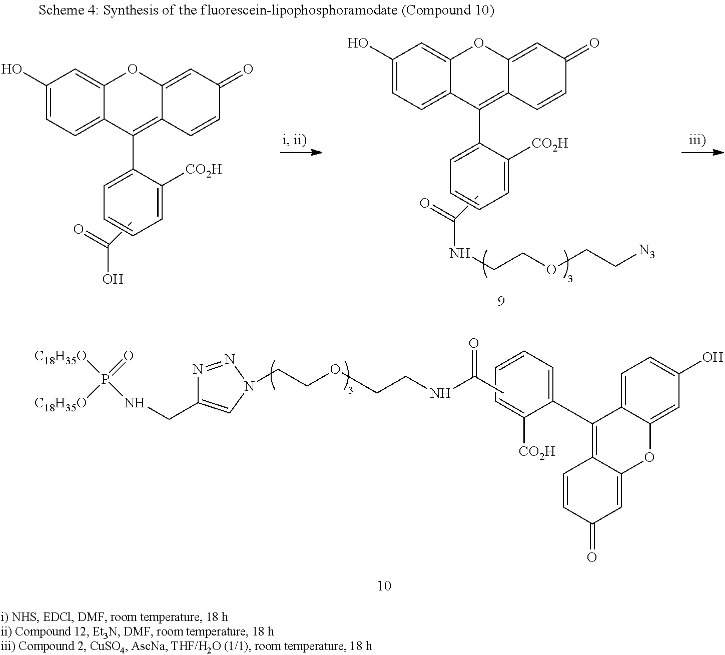

According to a second synthesis pathway, fluorescein can be attached to the lipophosphoramidate moiety by making use of the reverse click chemistry that involves for example one azide-lipophosphoramidate and one alkyne-fluorescein derivative. The azide-lipophosphoramidate (Compound 13) is readily obtained in a yield of 67% following an Atherton-Todd coupling that involves ω-amino-azido tetraethyleneglycol (Compound 12) and O,O-dioleylphosphite (Compound 1). Compound 13 is also a “clickable” building unit for the incorporation of lipid parts on a substrate according to the Huisgen coupling.

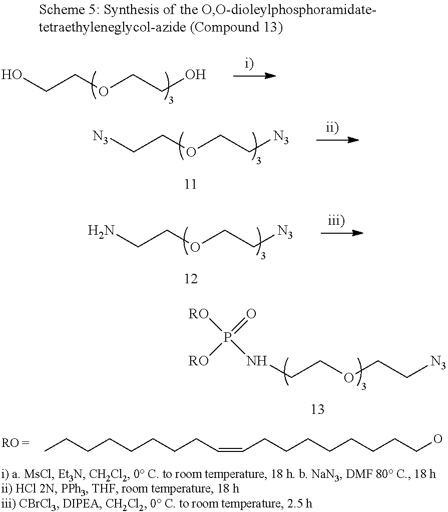

In a further step, the synthesis of the lipid-conjugate of fluorescein (Compound 15), that makes for example use of alkyne-fluorescein (Compound 14) synthesized following a procedure reported in M. D. Smith, D. Gong, C. G. Sudhahar, J. C. Reno, R. V. Stahelin, M. D. Best, Bioconjugate Chem., 2008, 19, 1855-1863, which is herein incorporated by reference, illustrates the use of the ω-azide-lipophosphoramidate (Compound 13) as depicted in Scheme 6 below. Also in that case, the Huisgen coupling was achieved with a good yield.

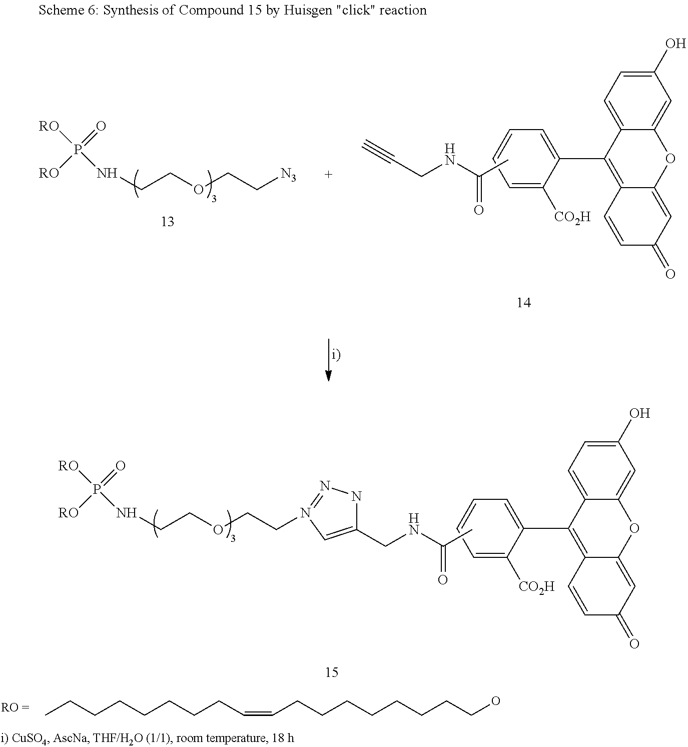

After the introduction of coumarin and fluorescein into the general structure defined in Figure 1, the incorporation of NBD (4-nitrobenzo[1,2,5]oxadiazole), a known fluorescent probe, has been investigated. Two synthesis pathways that involved either one azido-lipo-phosphoramidate plus one alkyne-NBD derivative or one alkyne-lipo-phosphoramidate plus one azide-NBD have been performed.

The synthesis of the NBD-based fluorescent phospholipids is shown in Schemes 7 to 9.

In a first synthesis pathway, in a preliminary step, the NBD fluorescent building block 43 was prepared by a nucleophilic aromatic substitution of a selected amine with 4-chloro-7-nitro-2,1,3-benzoxadiazole (4-chloro-NBD). Briefly, the alkyne-NBD 43 was prepared following a procedure that makes use of propargylamine as reactant. In a last step (Scheme 7), this “clickable” NBD fluorescent building block 43 was engaged in a Huisgen reaction.

Accordingly, the azide-lipophosphoramidate 13 was coupled with the alkyne-NBD 43 to produce the fluorescent lipid 44 in 80% yield.

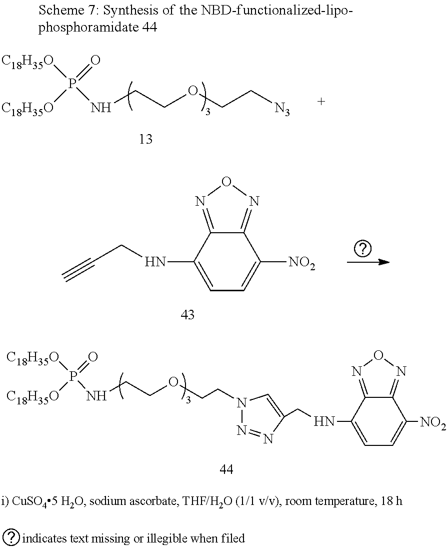

In a second synthesis pathway (Scheme 8), in a preliminary step, the NBD fluorescent building block 17 was prepared by a nucleophilic aromatic substitution of a selected amine with 4-chloro-7-nitro-2,1,3-benzoxadiazole (4-chloro-NBD). The synthesis of the azide-NBD building block 17 was prepared in 3 steps from 4-chloro-NBD as follows: an aromatic substitution of the halogen with 6-aminohexanoic acid followed by the activation of the carboxylic acid with NHS (N-hydroxysuccinimide) according to a Steglich's procedure (Compound 16) and, finally, the reaction of this activated ester with ω-amino-azido-tetraethyleneglycol 12. In a last step, this “clickable” NBD fluorescent building block 17 was engaged in a Huisgen reaction. Accordingly, the alkyne-lipophosphoramidate 2 was coupled to the azide-NBD building block 17 to produce the fluorescent phospholipid 18 (83% yield). In Compound 18, the NBD motif and the five methylene units, present between the NBD and the amide function, form a quite hydrophobic region. The presence of this pentyl chain allows to distance the fluorescent probe from the lipid chain.

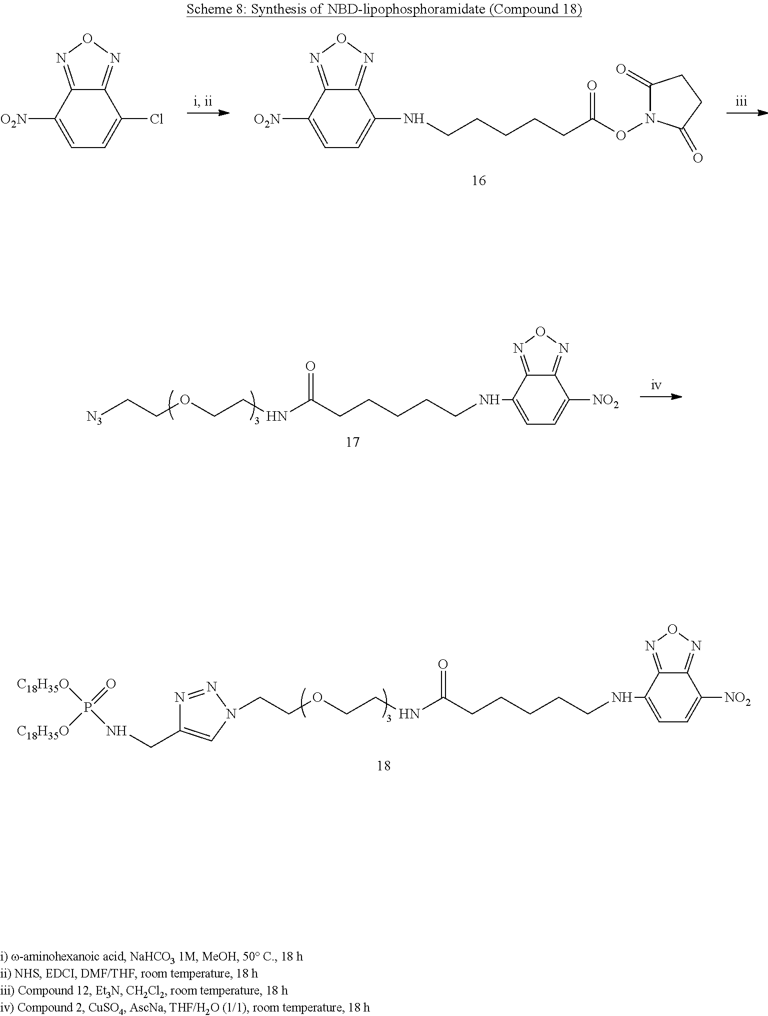

In a third synthesis pathway (Scheme 9), in a preliminary step, the NBD fluorescent building block 19 was prepared by a nucleophilic aromatic substitution of a selected amine with 4-chloro-7-nitro-2,1,3-benzoxadiazole (4-chloro-NBD). For the synthesis of the N3-functionalized NBD 19, the ω-amino-azido-tetraethyleneglycol 12 was engaged in the nucleophilic aromatic substitution. In a last step, this “clickable” NBD fluorescent building block 19 was engaged in a Huisgen reaction. Accordingly, the alkyne-lipophosphoramidate 2 was coupled to the azide-NBD building block 19 to produce the fluorescent phospholipid 20 (65% yield).

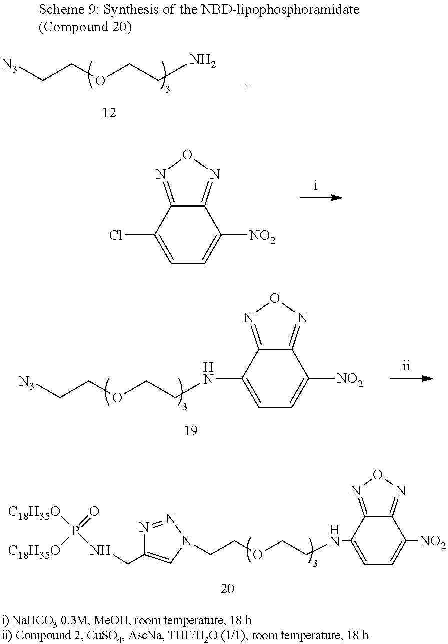

Naphthalimide can be also a fluorescent motif, for example if substituted with an amine in position 4. Naphthalimide exhibits an absorption at 400-440 nm and an emission at 500-550 nm. Two convergent synthesis routes were performed for the synthesis of naphthalimide functionalized lipo-phosphoramidates.

A first synthesis pathway is depicted in Scheme 10 below. The reaction of 4-bromo-1,8-naphthalic anhydride with ω-amino-azido-teraethyleneglycol 12 produced Compound 21. Next, the incorporation of the morpholine moiety was achieved to produce Compound 22. In the last step, the click reaction between the alkyne-lipophosphoramide 2 and Compound 22 produced Compound 23 in 78% yield.

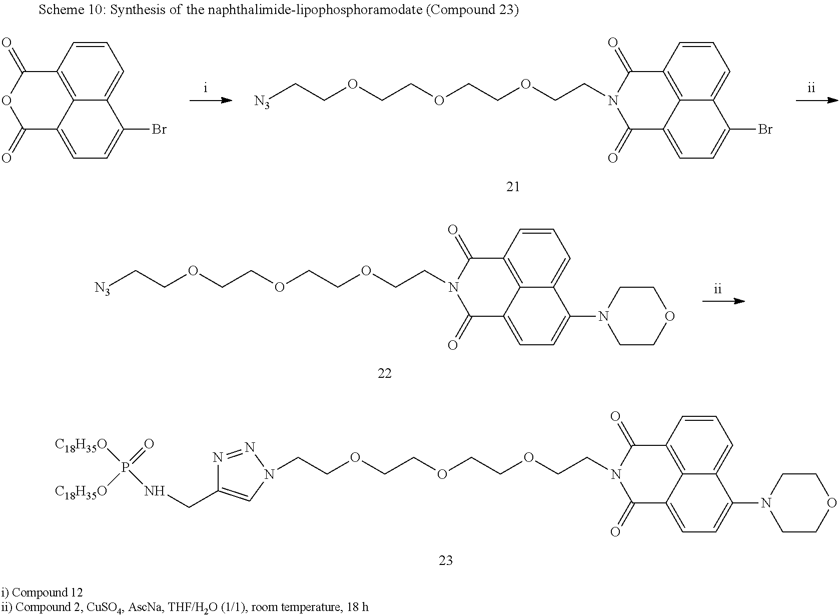

According to a second synthesis pathway (Scheme 11), propargylamine was first added to 4-bromo-1,8-naphthalic anhydride to produce Compound 45. Then, the introduction of the morpholine unit produced Compound 46 and the click coupling with the azido-lipo-phosphoramidate 13 produced the fluorescent lipophosphoramidate 47 in 85% yield.

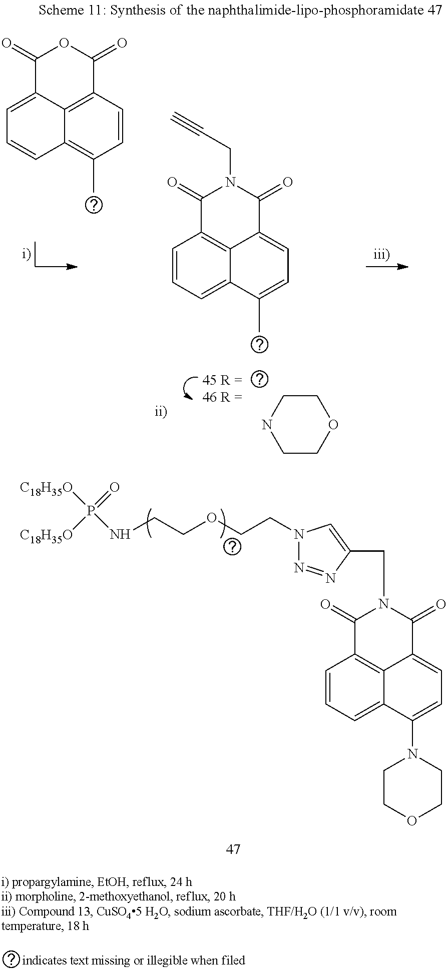

The synthesis of fluorescent lipophosphoramidates was achieved following a convergent synthesis scheme in which the last step consists in a Huisgen coupling of either one azide-lipophosphoramidate plus one alkyne fluorescent probe or one alkyne-lipophosphoramidate plus one azide-fluorescent group. With these fluorescent probes, lipids have been designed that exhibit fluorescent properties in complementary spectral domains. The synthesis scheme allows placing a linker of variable length on the phosphoramidate functional group on which two lipid chains and the fluorescent probe are attached.

Finally, with the aim of broadening the fluorescence excitation/emission domain of this series of fluorescent phospholipids, a cyanine-lipophosphoramidate 49 was synthesized. Cyanine motif is indeed a very interesting class of fluorescent probe with respect to their high fluorescent emission frequency that usually ranges between 700 to 800 nm. Such properties are particularly suitable for in vivo bio-distribution studies.

The synthesis of the cyanine labelled lipo-phosphoramidate 49 was achieved in a two steps procedure starting from the commercially available chlorocyanine IR-806 (Scheme 12). First, Compound 48 was prepared by the reaction of chlorocyanine IR-806 with propargylamine. In the last step, the alkyne-cyanine 48 was “clicked” with the azido-lipo-phosphoramidate 13. The cyanine-lipo-phosphoramidate 49, thus produced, was isolated, after purification, in 32% yield.

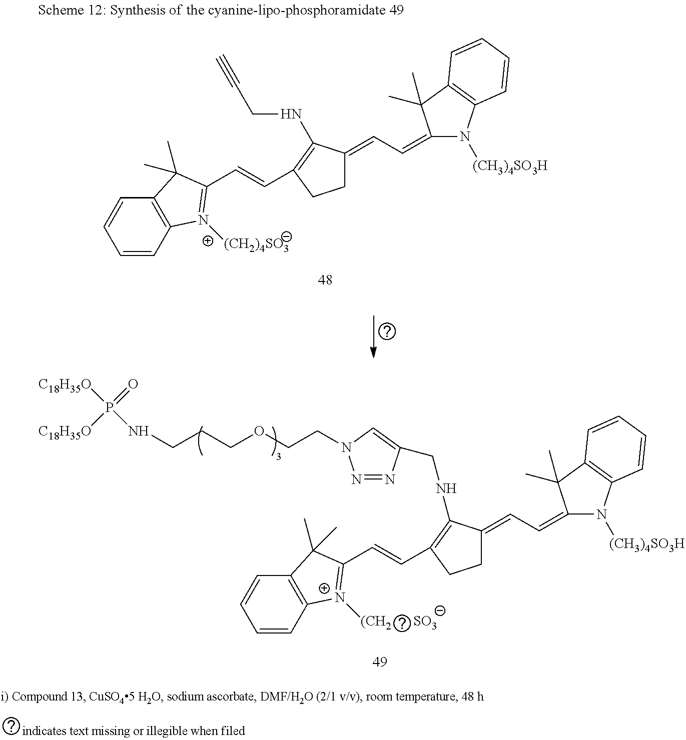

### EXAMPLES

Solvents were dried with a solvent purification system MBraun-SPS (THF, CH₂Cl₂) or freshly distilled on appropriate driers (DIPEA was distilled over NaOH). All compounds were fully characterized by ¹H¹ H (500.13 or 400.133 or 300.135 MHz), ¹³C (125.773 or 75.480 MHz) and ³¹P (161.970 or 121.498 MHz) NMR spectroscopy (Bruker AC 300, Avance DRX 400 and Avance DRX 500 spectrometers). Coupling constants J are given in Hertz. The following abbreviations were used: s for singlet, d doublet, t triplet, q quadruplet, qt quintuplet, m for multiplet, dd for doublet of doublets and dt for doublet of triplets. When needed, ¹³C heteronuclear HMQC and HMBC were used to unambiguously establish molecular structures. Mass spectroscopy analysis were performed by using a MS/MS high resolution Micromass ZABSpecTOF or on a Bruker Autoflex MALDI TOF-TOF III LRF200 CID. Oleyl alcohol 85% (Merck Schuchardt OHG, Germany) and other commercial compounds were used as received except DIPEA which was distilled over KOH. Dioleylphosphite 1 (V. Floch, M. P. Audrezet, C. Guillaume, E. Gobin, G. Le Bolch, J. C. Clement, J. J. Yaouanc, H. Des Abbayes, B. Mercier, J. P. Leroy, J. F. Abgrall, C. Férec, Biochim. Biophys. Acta 1998, 1371, 53-70.), Compound 14 (T. O. Harasym, P. Tardi, S. A. Longman, S. M. Ansell, M. B. Bally, P. R. Cullis, L. S. L. Choi, Bioconjugate Chem., 1995, 6, 187-194) Compound 16 (J. T. Elliott, G. D. Prestwich, Bioconjugate Chem., 2000, 11, 832-841) were synthesized following the methods reported in the documents between parentheses, which are herein incorporated by reference.

### Example 1

### Fluorescent Phospholipids—Phospholipid Building Blocs

### Synthesis of O,O-Dioleyl-N-propargylphosphoramidate (Compound 2)—Scheme 1

To a stirred solution of O,O-dioleylphosphite (1.0 g, 1.7 mmol) in dry CH₂Cl₂ (5 mL) was added propargylamine (95 mg, 1.7 mmol) under N₂ atmosphere. The mixture was cooled to 0° C. and CBrCl₃ (375 mg, 1.9 mmol) was added, followed by DIPEA (246 mg, 1.9 mmol). The reaction was allowed to warm up and stiffing continued for 2.5 h at room temperature. The reaction product was diluted with CH₂Cl₂ (50 mL), washed with water and brine, dried with MgSO₄, filtered and concentrated to furnish the Compound 2 as a yellow oil (yields: 92%). ¹H NMR (400 MHz, CDCl₃): δ 0.86 (6H, t, ³J=6.7 Hz), 1.26 (44H, m), 1.66 (4H, m, ³J=6.7 Hz), 2.01 (8H, m), 2.21 (1H, t, ³J=6.7 Hz), 2.80 (1H, m), 3.70 (2H, m), 4.02 (4H, q, ³J=6.7 Hz), 5.36 (4H, m). ³¹P NMR (162 MHz, CDCl₃): 8.13. ¹³C NMR (300 MHz, CDCl₃): 14.45, 23.02, 25.89, 27.55-32.94, 67.01, 71.65, 76.00, 130.12-130.32. HRMS (MALDI): m/z calc. for C₃₉H₇₄NO₃P [M+Na]: 658.53. Found: 658.453

### Synthesis of Phosphoramidate 7—Scheme 3

O,O-dioleylphosphite (Compound 1) (1.1 g, 1.9 mmol) and Compound 6 (420 mg, 1.9 mmol) were suspended in dry CH₂Cl₂ (5 mL) under nitrogen atmosphere. At 0° C., CBrCl₃ (397 mg, 2.1 mmol) was added followed by DIPEA (259 mg, 2.1 mmol). The reaction mixture was stirred for further 2.5 h while warmed to room temperature. The solvent was discarded and the residue was taken into Et₂O before DIPEA salts were separated. Column chromatography of the crude material with CH₂Cl₂/MeOH (95/5) produced Compound 7 as a colourless oil (yields: 67%). ¹H NMR (400 MHz, CDCl₃): δ 0.87 (6H, t, ³J=6.7 Hz), 1.26 (44H, m), 1.66 (4H, q, ³J=6.7 Hz), 1.98 (8H, m), 2.42 (1H, t, ³J=5.0 Hz), 3.07 (2H, m), 3.08 (1H, m, ³J=6.4 Hz), 3.53 (2H, t, ³J=5.0 Hz), 3.68 (12H, m), 3.98 (4H, q, ³J=6.4 Hz), 4.20 (2H, s), 5.35 (4H, m). ¹³C NMR (300 MHz, CDCl₃): 14.12, 22.68-32.61, 41.10, 64.37, 66.67, 69.73-71.22, 103.03, 110.31, 115.08, 119.09, 123.85, 129.76-130.44, 134.21, 144.78, 154.77, 156.30, 162.97. ³¹P NMR (162 MHz, CDCl₃): 9.78. HRMS (MALDI): m/z calc. for C₄₆H₉₁N₄O₇P [M⁺]: 811.645. Found: 811.829

### Synthesis of Phosphoramidate 13—Scheme 5

To a stirred solution of O,O-dioleylphosphite (1.2 g, 2.1 mmol) in dry CH₂Cl₂ (5 mL) under N₂ atmosphere was added Compound 12 (500 mg, 2.3 mmol). At 0° C., CBrCl₃ (456 mg, 2.3 mmol) was added followed by DIPEA (297 mg, 2.3 mmol) and the mixture was stirred for further 3.5 h at room temperature. The crude was diluted with CH₂Cl₂ (50 mL) and washed with water and brine, dried with MgSO₄, filtered and concentrated to furnish the Compound 13 as a yellow oil (yields: 67%). ¹H NMR (400 MHz, CDCl₃): δ 0.86 (6H, t, ³J=6.7 Hz), 1.26 (44H, m), 1.66 (4H, q, ³J=6.7 Hz), 1.98 (8H, m), 3.08 (1H, m), 3.40 (2H, t, ³J=6.4 Hz), 3.52-3.69 (12H, m), 3.98 (4H, q, ³J=6.4 Hz), 5.35 (4H, m). ¹³C NMR (300 MHz, CDCl₃): 14.45, 23.06-32.95, 41.45, 51.02, 66.72, 70.42-71.64, 130.12-130.31. ³¹P NMR (162 MHz, CDCl₃): 9.78.

### Example 2

### Fluorescent Phospholipids—Click Reactions Involving Phospholipid Building Blocks and Fluorophores

### Synthesis of Phosphoramidate 4a—Scheme 2

Phosphoramidate 2 (200 mg, 0.31 mmol) and Compound 3a (64 mg, 0.31 mmol) were suspended in THF (5 mL). CuSO₄.5H₂O (77 mg, 0.31 mmol) and sodium ascorbate (62 mg; 0.31 mmol) were added along with water and the mixture was stirred in the dark for 18 h. The organic solvent was discarded and the aqueous layer was extracted twice with CH₂Cl₂. The combined organic layers were washed with water and brine, dried with MgSO₄, filtered and concentrated. Column chromatography of the residue with EtOAc/Hexane (5/1) furnished the Compound 4a as a yellow oil (yields: 91%). ¹H NMR (400 MHz, CDCl₃): δ 0.86 (6H, t, ³J=6.7 Hz), 1.26 (44H, m), 1.66 (4H, qt, ³J=6.7 Hz), 1.99 (8H, m), 3.58 (1H, m, ³J=6.4 Hz), 4.05 (4H, q, ³J=6.4 Hz), 4.35 (1H, m, ³J=8.8), 5.34 (4H, m), 6.86 (1H, d, ³J=8.8 Hz), 6.89 (1H, s), 7.40 (1H, d, ³J=8.8 Hz), 8.33 (1H, s), 8.45 (1H, s). ¹³C NMR (300 MHz, CDCl₃): 14.41, 23.09-32.93, 37.22, 67.79, 103.05, 110.72, 115.51, 119.57, 130.08-130.53, 134.43, 155.04, 156.66, 163.18. ³¹P NMR (162 MHz, CDCl₃): 8.70. HRMS (MALDI): m/z calc. for C₄₈H₇₉N₄O₆P [M⁺]: 838.574. Found: 838.684. (λ_{max,exc}=349 and 412 nm (EtOH); λ_{max,em}=425 and 479 nm (EtOH).

### Synthesis of Phosphoramidate 4b—Scheme 2

Phosphoramidate 2 (150 mg, 0.24 mmol) and Compound 3b (48 mg, 0.24 mmol) were suspended in THF (5 mL). CuSO₄.5H₂O (59 mg, 0.24 mmol) and sodium ascorbate (47 mg, 0.24 mmol) were added along with water and the mixture was stirred in the dark for 18 h. Organic solvent was discarded and the aqueous layer was extracted twice with CH₂Cl₂. The combined organic layers were washed with water and brine, dried with MgSO₄, filtered and concentrated. Column chromatography of the residue with EtOAc/Hexane (100/0-60/40 v/v) produced Compound 4b as a yellow oil; yield 148 mg (72%). ¹H NMR (400 MHz, CDCl₃): δ 0.86 (t, J=6.7 Hz, 6H, CH₃), 1.25 (m, 44H, CH₂), 1.65 (q, J=6.7 Hz, 4H, CH₂), 1.98 (m, 8H, CH₂), 3.22 (m, 1H, NH), 3.91 (s, 3H, OCH₃), 3.99 (q, J=6.4 Hz, 4H, CH₂OP), 4.27 (m, 2H, NCH), 5.31 (m, 4H, CH═CH), 6.89 (s, 1H, Ar—H), 6.94 (d, 1H, Ar—H), 7.53 (d, J=8.8 Hz, 1H, CH═CH), 8.49 (s, 1H, Ar—H), 8.55 (s, 1H, H-triazol). ¹³C NMR (125 MHz, CDCl₃): 14.1, 22.7-32.6, 38.9, 66.7, 100.7, 111.5, 114.1, 122.6, 120.2, 129.8-130.41, 133.7 146.85, 154.7, 156.0, 163.8. ³¹P NMR (162 MHz, CDCl₃): 8.9. MS (MALDI): m/z calcd. for C₄₉H₈₁N₄O₆P [(M+H)⁺]: 852.589. Found: 853.604. (λ_{max,exc}=344 nm (EtOH); λ_{max,em}=416 nm (EtOH).

### Synthesis of Phosphoramidate 8—Scheme 3

Phosphoramidate 7 (150 mg, 0.20 mmol) and Compound 3a (73 mg, 0.20 mmol, 1.0 equiv) were combined in THF (2.5 mL). CuSO₄ and sodium ascorbate (40 mg, 0.20 mmol) were added along with water (2.5 mL) and the mixture was stirred in the dark for 18 h. After concentration, the aqueous layer was extracted twice with CH₂Cl₂. The organic layer was washed with water and brine, dried with MgSO₄, filtered and concentrated. The residue was purified by column chromatography with AcOEt/MeOH (30/0-30/1) as eluent. Compound 8 was isolated as a yellow oil (yields: 80%). δ 0.86 (6H, t, ³J=6.7 Hz), 1.26 (44H, m), 1.66 (4H, q, ³J=6.7 Hz), 2.01 (8H, m), 3.07 (2H, m), 3.39 (1H, m, ³J=6.4 Hz), 3.52 (2H, t, ³J=6.4 Hz), 3.65-3.74 (12H, m), 3.92 (4H, q, ³J=6.4 Hz), 4.74 (2H, s), 5.36 (4H, m), 6.88 (1H, s, ³J=8.8 Hz), 6.90 (1H, s), 7.39 (1H, d, ³J=8.8 Hz), 8.37 (1H, s), 8.52 (1H, s). ¹³C NMR (300 MHz, CDCl₃): 14.12, 22.68-32.61, 41.10, 64.37, 66.67, 69.73-71.22, 103.03, 110.31, 115.08, 119.09, 123.85, 129.76-130.44, 134.21, 144.78, 154.77, 156.30, 162.97. ³¹P NMR (162 MHz, CDCl₃): 9.63. HRMS (MALDI): m/z calc. for C₅₆H₉₅N₄O₁₀P [M+H⁺]: 1014.679. Found: 1014.602. (λ_{max,exc}=345 and 411 nm (EtOH); λ_{max,em}=425 and 478 nm (EtOH).

### Synthesis of Compounds 9 and 10—Scheme 4

NHS-carboxyfluorescein (200 mg, 0.42 mmol), Compound 12 (92 mg, 0.42 mmol) and Et₃N (128 mg, 1.27 mmol) were added together in dry DMF (2.5 mL). The solution was stirred at room temperature for 18 h. The solvent was removed under reduced pressure and the residue was purified by column chromatography DCM/MeOH/AcOH (27/3/0.3) to give Compound 9 as an orange oil (yields: 85%). ¹H NMR (400 MHz, CD₃OD): δ 3.69-3.75 (m), 6.64 (m), 6.70 (m,), 6.79 (m), 7.38 (m), 7.75 (m), 8.06 (bs), 8.24 (m), 8.30 (m), 8.53 (m).

Phosphoramide 2 (220 mg, 0.35 mmol) and Compound 9 (200 mg, 0.35 mmol) were reacted in THF (2.5 mL). CuSO₄ (87 mg, 0.35 mmol) and sodium ascorbate (69 mg, 0.35 mmol) were added along with water and the mixture was stirred in the dark for 18 h. After concentration, the aqueous layer was extracted twice with CH₂Cl₂. The organic layer was washed with water and brine, dried with MgSO₄, filtered and concentrated. The residue was purified by column chromatography CH₂Cl₂/MeOH (9/1) to give Compound 10 as an orange oil (yields: 84% mixture of isomers). HRMS (MALDI): m/z calc. for C₆₈H₁₀₂N₅O₁₂P [M+H⁺]: 1213.734. Found: 1212.772. ¹H NMR (400 MHz, CDCl₃-CD₃OD, 2/1 v/v): δ=0.6 (t, CH₃), 0.97 (m, CH₂), 1.30 (q, CH₂), 1.68 (m, CH₂), 2.97 (bs, NH), 3.17 (m, NHCH₂), 3.28-3.62 (m, CH₂-TEG), 3.82 (m, NCH₂), 4.14 (q, CH₂OP), 5.04 (m, CH═CH), 6.21 (m, ═CH), 6.38 (m, Ar—H), 6.96 (d, Ar—H), 7.34-7.51 (m, Ar—H), 8.14 (s, H-triazol). ³¹P NMR (162 MHz, CDCl₃-CD₃OD, 2/1 v/v): 9.4. (λ_{max,exc}=505 nm (EtOH); λ_{max,em}=527 nm (EtOH); εₘₐₓ=15700).

### Synthesis of Phosphoramidate 15—Scheme 6

Phosphoramidate 13 (200 mg, 0.31 mmol) and Compound 14 (64 mg, 0.31 mmol) were suspended in THF (5 mL). CuSO₄ and sodium ascorbate (62 mg, 0.31 mmol) were added along with water and the mixture was stirred in the dark for 18 h. After concentration, the aqueous layer was extracted twice with CH₂Cl₂. The organic layer was washed with water and brine, dried with MgSO₄, filtered and concentrated. Column chromatography of the residue gave Compound 15 as a yellow oil.

### Synthesis of Compounds 43 and 44—Scheme 7

To a solution of NBD-Cl (500 mg, 2.51 mmol) in dry CH₃CN (20 mL) was added propargylamine (276 mg, 0.32 mmol) and the reaction mixture was stirred for 2 h at room temperature. The solvent was evaporated and column chromatography of the brown residue with hexane/EtOAc (3/1.5 v/v) produced Compound 43 as a brown solid; yield: 164 mg (30%). ¹H NMR (400 MHz, CDCl₃): δ=2.31 (t, J=2.4 Hz, 1H, C≡CH), 3.80, (s, 2H, CH₂C≡CH), 4.11 (d, J=2.4 Hz 1H, NH), 6.18 (d, J=8.7 Hz, 1H, Ar—H), 8.34 (d, J=8.7 Hz, 1H, Ar—H)

Phosphoramide 13 (220 mg, 0.35 mmol) and Compound 43 (200 mg, 0.35 mmol) were reacted in THF (2.5 mL). CuSO₄.5H₂O (87 mg, 0.35 mmol) and sodium ascorbate (69 mg, 0.35 mmol) were added along with water and the mixture was stirred in the dark for 20 h. After concentration, the aqueous layer was extracted twice with CH₂Cl₂. The organic layer was washed with water and brine, dried with MgSO₄, filtered and concentrated. The residue was purified by column chromatography CHCl₃/MeOH (30/1 v/v) to give Compound 44 as an orange oil; yield 285 mg (80%). ¹H NMR (400 MHz, CDCl₃): δ=0.86 (t, J=6.7 Hz, 6H, CH₃), 1.26 (m, 44H, CH₂), 1.63 (q, J=6.7 Hz, 4H, CH₂), 2.01 (m, 8H, CH₂), 3.06 (m, 2H, NHCH₂), 3.59-3.71 (m, 14H, CH₂-TEG), 3.84 (t, J=6.4 Hz, 2H, NHCH₂CH₂), 3.96 (q, J=6.4 Hz, 4H, CH₂OP), 4.56 (t, J=6.4 Hz, 2H, CH₂NH), (m, 4H, CH═CH), 6.38 (d, J=8.0 Hz, 1H, ═CH), 7.90 (s, 1H, H-triazol), 8.45 (d, J=8.0 Hz, 1H, ═CH), 8.29 (bs, 1H, NH). ¹³C NMR (175 MHz, CDCl₃): 14.1, 22.7-32.6, 37.0, 50.3, 66.7, 68.3, 69.4-70.6, 129.7-130.4, 133.5, 136.5, 144.4. ³¹P NMR (162 MHz, CDCl₃): 9.5. HRMS (MALDI): m/z calcd. for C₅₃H₉₅N₈O₉P [(M+Na)⁺]: 1039.670. Found: 1039.632. (λ_{max,exc}=453 nm (EtOH); λ_{max,em}=525 nm (EtOH); εₘₐₓ=14900).

### Synthesis of Compounds 17 and 18—Scheme 8

Compound 16 (300 mg, 0.77 mmol) and Compound 12 were mixed in dry CH₂Cl₂ (10 mL) and Et₃N (234 mg, 2.31 mmol) was added. The reaction was stirred at room temperature for 18 h. The solvent was evaporated under reduced pressure and column chromatography with AcOEt/MeOH (30/1-30/2) produced Compound 17 as an orange oil (yields: 88%). ¹H NMR (400 MHz, CDCl₃): δ 1.25 (2H, m), 1.50 (2H, m), 1.72 (2H, m), 1.84 (2H, m), 2.24 (2H, m), 3.37 (2H, m), 3.45 (2H, m), 3.54 (4H, m), 3.62 (8H, m), 4.08 (2H, m), 6.15 (1H, m, ³J=8.0 Hz), 6.25 (1H, m), 6.98 (1H, bs), 8.45 (1H, dd, ³J=4.0 Hz). ¹³C NMR (300 MHz, CDCl₃): 25.02, 25.81, 26.63, 28.19, 39.61, 44.03, 51.01, 70.11-70.92, 98.92, 137.00, 144.63, 144.63, 173.54. HRMS (MALDI): m/z calc. for C₂₀H₃₀N₈O₇ [M⁺]: 494.224. Found: 494.434

Phosphoramide 2 (100 mg, 0.16 mmol) and Compound 17 (73 mg, 0.16 mmol) were mixed in THF (2.5 mL). CuSO₄ (40 mg, 0.16 mmol) and sodium ascorbate (32 mg, 0.16 mmol) were added along with water (2.5 mL) and the mixture was stirred in the dark for 18 h. After concentration, the aqueous layer was extracted twice with CH₂Cl₂. The organic layer was washed with water and brine, dried with MgSO₄, filtered and concentrated. The residue was purified by column chromatography with AcOEt/MeOH (30:1-30:0) to give Compound 18 as an orange oil (yields: 83%). ¹H NMR (400 MHz, CDCl₃): δ 0.86 (6H, t, ³J=6.7 Hz), 1.26 (44H, m), 1.50 (2H, q, ³J=8.0 Hz), 1.63 (4H, m, ³J=6.7 Hz), 1.71 (2H, qt, ³J=8.0 Hz), 1.82 (2H, qt, ³J=8.0 Hz), 2.01 (8H, m), 2.21 (1H, t, ³J=6.7 Hz), 2.24 (2H, t, ³J=8.0 Hz), 3.42 (2H, m), 3.54-3.58 (12H, m), 3.70 (1H, m), 3.75 (2H, m), 4.02 (4H, q, ³J=6.4 Hz), 4.23 (2H, m), 4.53 (2H, m), 5.36 (4H, m), 6.15 (1H, d, ³J=8.0 Hz), 6.85 (1H, bs), 7.5 (1H, bs), 7.78 (1H, s), 8.46 (1H, d, ³J=8.0 Hz). ¹³C NMR (300 MHz, CDCl₃): 14.13, 22.69-32.63, 35.76, 37.05, 39.12, 44.01, 50.31, 66.75, 69.38-70.58, 122.25, 129.74-130.48, 144.11, 144.37, 173.14. ³¹P NMR (162 MHz, CDCl₃): 7.60. HRMS (MALDI): m/z calc. for C₅₉H₁₀₄N₉O₁₀P [M+Na]: 1152.754. Found: 1152.726. (λ_{max,exc}=466 nm (EtOH); λ_{max,em}=530 nm (EtOH); εₘₐₓ=19300).

### Synthesis of Compounds 19 and 20—Scheme 9

To Compound 12 (790 mg, 4.14 mmol) in 10 mL of NaHCO₃ 0.3 M was added 7-chloro-4-nitrobenzofurazan (500 mg, 3.33 mmol) in 20 mL of MeOH and the reaction was stirred at room temperature for 18 h. The crude was concentrated and the brown residue was submitted to column chromatography with AcOEt/hexane (2/1). Compound 19 was isolated as brown oil (yields: 40%). ¹H NMR (400 MHz, CDCl₃): δ 3.38 (2H, t, ³J=4.8 Hz), 3.65-3.72 (12H, m), 3.87 (2H, t, ³J=4.8 Hz), 6.19 (1H, d, ³J=8.8 Hz), 7.10 (1H, bs), 8.47 (1H, d, ³J=8.8 Hz). ¹³C NMR (300 MHz, CDCl₃): 43.77, 50.68, 68.14, 69.99-70.70, 81.59, 98.78, 136.50, 144.22.

Phosphoramide 2 (75 mg, 0.12 mmol) and Compound 17 (45 mg, 0.12 mmol) were mixed in THF (2.5 mL). CuSO₄ (40 mg, 0.16 mmol) and sodium ascorbate (32 mg, 0.16 mmol) were added along with water (2.5 mL) and the mixture was stirred in the dark for 18 h. After concentration, the aqueous layer was extracted twice with CH₂Cl₂. The organic layer was washed with water and brine, dried with MgSO₄, filtered and concentrated. The residue was purified by column chromatography with AcOEt/MeOH (30/1-30/2) to give Compound 20 as an orange oil (yields: 65%). ¹H NMR (400 MHz, CDCl₃): δ 0.86 (6H, t, ³J=6.7 Hz), 1.26 (44H, m), 1.63 (4H, m, ³J=6.7 Hz), 2.01 (8H, m), 3.58 (1H, bs), 3.59-3.71 (10H, m), 3.86 (4H, m), 3.97 (4H, q, ³J=6.4 Hz), 4.21 (2H, t, ³J=4.0 Hz), 4.51 (2H, t, ³J=4.0 Hz), 5.36 (4H, m), 6.19 (1H, d, ³J=8.0 Hz), 7.70 (1H, bs), 7.71 (1H, s), 8.47 (1H, d, ³J=8.0 Hz). ¹³C NMR (300 MHz, CDCl₃): 14.12, 22.69-32.63, 37.00, 50.34, 66.75, 68.30, 69.36-70.57, 129.72-130.45, 133.50, 136.50, 144.38. ³¹P NMR (162 MHz, CDCl₃): 8.44. HRMS (MALDI): m/z calc. for C₅₉H₁₀₄N₉O₁₀P [M+H]: 1016.680. Found: 1017.697. (λ_{max,exc}=462 nm (EtOH); λ_{max,em}=535 nm (EtOH); εₘₐₓ=13200).

### Synthesis of Compounds 21, 22 and 23—Scheme 10

4-bromo-1,8-naphthalene dicarboxylic anhydride (1.5 g, 5.4 mmol, 1.0 equiv) and 1-amino-11-azido-3,6,9-trioxaundecane (1.3 g, 6.5 mmol, 1.2 equiv) were refluxed in absolute EtOH (50 mL) for 18 h. The solvent was discarded and column chromatography of the residue with AcOEt/hexane (1.5/1) gave Compound 21 as colourless crystals (yields: 50%). ¹H NMR (400 MHz, CDCl₃): δ 3.32 (2H, t, ³J=5.2 Hz), 3.52 (8H, m), 3.66 (2H, t, ³J=2.4 Hz), 3.78 (2H, t, ³J=6.0 Hz), 4.37 (2H, m, ³J=6.4 Hz), 7.73 (1H, t, ³J=7.2 Hz), 7.92 (2H, d, ³J=7.6 Hz), 8.30 (1H, d, ³J=8.0 Hz), 8.35 (1H, d, ³J=7.6 Hz), 8.44 (1H, d, ³J=7.6 Hz). ¹³C NMR (300 MHz, CDCl₃): 26.44, 39.13, 50.55, 67.74, 69.88-70.53, 81.54, 121.96, 122.81, 127.91-133.05, 163.36.

Compound 21 (500 mg, 1.0 mmol) and morpholine (91 mg, 1.0 mmol) were refluxed in absolute 2-methoxyethanol (20 mL) for 18 h. The solvent was discarded and column chromatography of the residue with DCM/MeOH (10/0.5) gave Compound 22 as a colourless oil (yields: 98%). ¹H NMR (400 MHz, CDCl₃): δ 3.32 (4H, t, ³J=5.2 Hz), 3.39 (2H, t, ³J=7.6 Hz), 3.64.3.74 (10H, m, ³J=2.4 Hz), 3.80 (2H, t, ³J=6.4 Hz), 4.00 (4H, t, ³J=4.4 Hz), 4.42 (2H, t, ³J=8.0 Hz), 7.23 (1H, d, ³J=7.6 Hz), 7.70 (1H, t, ³J=8.0 Hz), 8.41 (1H, d, ³J=7.6 Hz), 8.51 (1H, d, ³J=7.6 Hz), 8.57 (1H, d, ³J=7.6 Hz). ¹³C NMR (MHz, CDCl₃): 29.20, 38.98, 50.64, 53.41, 66.94, 67.96, 69.93, 70.13, 70.62, 114.92, 117.10, 123.27, 125.81, 126.12, 129.92-130.07, 131.19, 132.54, 155.63, 163.93, 164.41. HRMS (MALDI): m/z calc. for C₂₄H₂₉N₅O₆ [M+H]: 476.070. Found: 477.134.

Phosphoramide 2 (100 mg, 0.16 mmol) and Compound 22 (76 mg, 0.16 mmol) were mixed in THF (2.5 mL). CuSO₄ (39 mg, 0.16 mmol) and sodium ascorbate (31 mg, 0.16 mmol) were added along with water (2.5 mL) and the mixture was stirred in the dark for 18 h. After concentration, the aqueous layer was twice extracted with CHCl₃. The organic layer was washed with water and brine, dried with MgSO₄, filtered and concentrated. The residue was purified by column chromatography CHCl₃/MeOH (28/2) to give Compound 23 as an orange oil (yield: 78%). ¹H NMR (400 MHz, CDCl₃): δ 0.87 (4H, t, ³J=6.7 Hz), 1.25 (44H, m), 1.61 (4H, m), 1.99 (8H, m), 3.62 (4H, t, ³J=5.2 Hz), 3.48.3.60 (6H, m), 3.69 (2H, t, ³J=4.8 Hz), 3.80 (4H, q, ³J=5.6 Hz), 3.96 (4H, t, ³J=4.4 Hz), 4.02 (4H, m), 4.20 (2H, t, ³J=6.8 Hz), 4.41 (2H, t, ³J=6.8 Hz), 4.47 (2H, t, ³J=6.0 Hz), 5.33 (4H, m), 7.23 (1H, d, ³J=7.6 Hz), 7.67 (1H, s), 7.70 (1H, t, ³J=8.0 Hz), 8.41 (1H, d, ³J=7.6 Hz), 8.51 (1H, d, ³J=7.6 Hz), 8.57 (1H, d, ³J=7.6 Hz). ¹³C NMR (MHz, CDCl₃): 14.11, 22.66-32.59, 37.01, 39.01, 50.24, 53.43, 66.49, 66.55, 66.95, 67.98, 69.42, 70.18, 70.53, 114.95, 116.99, 123.20, 125.84, 126.11, 129.77-130.16, 131.22, 132.59, 155.70, 163.93, 164.41. ³¹P NMR (162 MHz, CDCl₃): 8.88. HRMS (MALDI): m/z calc. for C₅₉H₁₀₄N₉O₁₀P [M+H]: 1118.752. Found: 1119.777. (λ_{max,exc}=436 nm (EtOH); λ_{max,em}=532 nm (EtOH); εₘₐₓ=7500).

### Synthesis of Compounds 45, 46 and 47—Scheme 11

To a solution of 4-bromo-1,8-naphthalic anhydride (1.0 g, 3.61 mmol) in absolute EtOH (20 mL) was added propargylamine (240 mg, 4.33 mmol). The reaction was refluxed for 20 h. The reaction was cooled down to room temperature and filtered. The filtrate was concentrated in vacuum and the residue purified by flash column chromatography with hexane/EtOAc (1/1-1/2 v/v) to produce Compound 45 as a yellow solid; yield 960 mg (85%). ¹H NMR (400 MHz, CDCl₃): δ=2.06 (t, J=2.4 Hz, 1H, C≡CH), 1.26 (s, 2H, CH₂C≡CH), 7.04 (d, J=8.1 Hz, 1H, Ar—H), 7.55 (t, J=8.1 Hz, 1H, Ar—H), 8.24 (d, J=8.1 Hz, 1H, Ar—H), 8.30 (d, J=8.1 Hz, 1H, Ar—H), 8.36 (d, J=8.1 Hz, 1H, Ar—H)

Compound 45 (300 mg, 0.96 mmol) and morpholine (420 mg, 4.80 mmol) were dissolved in 2-methoxyethanol (20 mL) and the mixture was refluxed overnight. The solvent was evaporated under reduced pressure and flash column chromatography of the residue with CH₂Cl₂/MeOH (95/5 v/v) furnished Compound 46 as yellow crystals; yield 292 mg (95%). ¹H NMR (400 MHz, CDCl₃): δ=2.06 (t, J=2.5 Hz, 1H, C≡CH), 3.11 (m, J=4.5 Hz, 4H, CH₂), 3.80 (m, J=4.5 Hz, 4H, CH₂), 4.68 (s, 2H, CH₂C≡CH), 7.04 (d, J=8.1 Hz, 1H, Ar—H), 7.55 (t, J=8.1 Hz, 1H, Ar—H), 8.24 (d, J=8.1 Hz, 1H, Ar—H), 8.30 (d, J=8.1 Hz, 1H, Ar—H), 8.36 (d, J=8.1 Hz, 1H, Ar—H). ¹³C NMR (75 MHz, CDCl₃): 29.0, 53.1, 66.6, 70.6, 78.2, 114.8, 116.1, 122.4-132.9, 156.0, 163.2, 163.7. HRMS (MALDI): m/z calc. for C₁₉H₁₆N₂O₃ [(M+H)⁺]: 321.124. Found: 321.069

Phosphoramide 13 (100 mg, 0.31 mmol) and Compound 46 (250 mg, 0.31 mmol) were reacted in THF (2.5 mL). CuSO₄.5H₂O (87 mg, 0.31 mmol) and sodium ascorbate (78 mg, 0.31 mmol) were added along with water (2.5 mL) and the mixture was stirred in the dark for 18 h. After concentration, the aqueous layer was extracted twice with CH₂Cl₂. The organic layer was washed with water and brine, dried with MgSO₄, filtered and concentrated. The residue was purified by column chromatography CHCl₃/MeOH (30/1 v/v) to give Compound 47 as an orange oil; yield 295 mg (85%). ¹H NMR (400 MHz, CDCl₃): δ=0.87 (t, J=6.7 Hz, 6H, CH₃), 1.25 (m, 44H, CH₂), 1.61 (q, J=6.7 Hz, 4H, CH₂), 1.99 (m, 8H, CH₂), 3.04 (m, 2H, CH₂), 3.10 (m, 1H, NH), 3.25 (m, 4H, CH₂), 3.50-3.55 (m, 12H, CH₂-TEG), 3.80 (t, 2H, J=6.0 Hz, CH₂), 3.99 (m, 4H, CH₂), 4.01 (m, 4H, CH₂OP), 4.46 (t, J=6.0 Hz, 2H, (m, 4H, CH₂), 5.33 (m, 4H, CH═CH), 7.20 (d, J=7.6 Hz, 1H, Ar—H), 7.68 (t, J=7.6 Hz, 1H, Ar—H), 7.76 (s, 1H, H-triazol), 8.39 (d, J=7.6 Hz, 1H, Ar—H), 8.57 (d, J=7.6 Hz, 1H, Ar—H), 8.59 (d, J=7.6 Hz, 1H, Ar—H). ¹³C NMR (125 MHz, CDCl₃): 14.4, 23.0-35.5, 41.4, 50.8, 53.8, 66.7, 67.3, 70.7, 70.8, 71.7, 115.3, 117.3, 123.5, 125.6, 126.2, 129.4-133.1, 143.9, 156.1, 163.9, 164.4. ³¹P NMR (162 MHz, CDCl₃): 9.8. HRMS (MALDI): m/z calcd. for C₆₃H₁₀₃N₆O₉P [(M+Na)⁺]: 1141.742. Found: 1141.709. (λ_{max,exc}=406 nm (EtOH); λ_{max,em}=532 nm (EtOH); εₘₐₓ=10700).

### Synthesis of Compound 49—Scheme 12

Phosphoramide 13 (55 mg, 0.07 mmol) and Compound 48 (50 mg, 0.07 mmol) were combined in a (2/1) mixture of DMF (2.5 mL) and water. CuSO₄.5H₂O (17 mg, 0.07 mmol) and sodium ascorbate (13.5 mg, 0.07 mmol) were added and the mixture was stirred in the dark for 48 h. The crude mixture was diluted with 5 mL of water and extracted several times with CH₂Cl₂. The organic layers were combined, washed with water, dried with MgSO₄, filtered and concentrated. The residue was purified by column chromatography CH₂Cl₂/MeOH (9/1-7/1 v/v) to give Compound 49 as an dark blue oil; yield 295 mg (32%). ¹H NMR (400 MHz, DMSO-d₆/CDCl₃): δ=0.82 (t, J=6.7 Hz, 6H, CH₃), 1.20 (m, 44H, CH₂), 1.40 (s, 12H, CH₃), 1.51 (m, 8H, CH₂), 1.72 (q, 4H, CH₂), 1.94 (m, 8H, CH₂), 2.72 (m, 4H, CH₂), 2.82 (m, 2H, CH₂), 3.34 (s, 4H, CH₂), 3.43-3.55 (m, 12H, CH₂-TEG), 3.79 (m, 4H, CH₂), 3.89 (m, 4H, CH₂OP), 4.52 (m, 2H, CH₂), 4.68 (m, 1H, NH), 4.96 (m, 2H, CH₂), 5.26 (m, 4H, CH═CH), 5.52 (d, J=12.4 Hz, 2H, CH₂), 6.98 (m, 2H, ═CH), 7.08 (m, 2H, ═CH), 7.22 (m, 2H, ═CH), 7.30 (m, 2H, ═CH), 7.75 (m, 2H, ═CH), 8.13 (s, 1H, H-triazol). ¹³C NMR (125 MHz, DMSO-d₆/CDCl₃): 14.4, 22.1, 25.1, 26.6, 28.0-31.4, 32.0, 40.3, 47.1, 50.1, 53.0, 66.1, 66.2-72.8, 76.9, 96.3, 109.0, 122.5, 123.5, 126.5, 128.2, 129.2-133.1, 134.4, 139.9, 140.9, 143.90, 165.9, 167.7. ³¹P NMR (162 MHz, CDCl₃): 9.8. HRMS (MALDI): m/z calcd. for C₈₄H₁₃₆N₇O₁₂PS₂ [(M+K)⁺]: 1568.906. Found: 1569.020

### Synthesis of Compounds 24, 25, 26, 27a and 27b

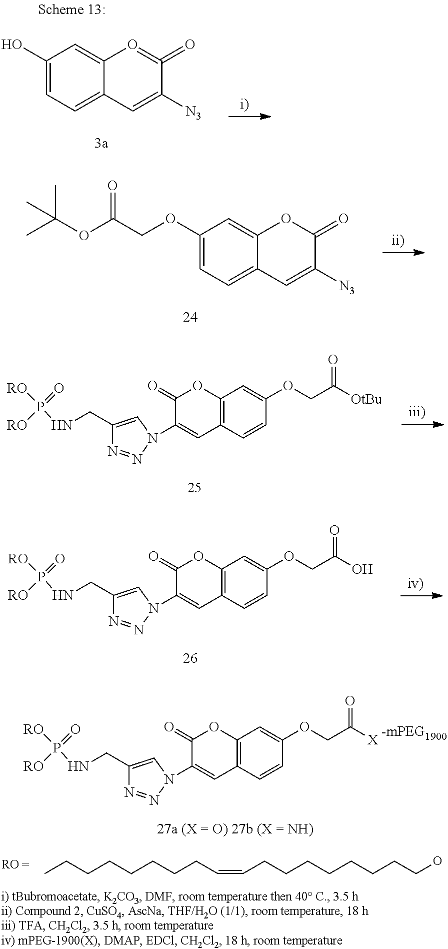

3-Azido-7-hydroxy-coumarin (Compound 3a) (500 mg, 0.25 mmol), and K₂CO₃ (73 mg, 0.30 mmol) were suspended in dry DMF (6 mL) under nitrogen atmosphere and stirred at room temperature for 10 minutes. The mixture was heated at 40° C. and tert-butyl-2-bromoacetate (50 mg, 0.29 mmol) was added. The mixture was heated for further 3.5 hours. The crude was concentrated and the residue diluted with AcOEt (100 mL). The organic layer was washed with saturated aqueous NH₄Cl. The aqueous phase was further extracted with AcOEt (2×50 mL) and the combined organic phases were dried with MgSO₄, filtered and concentrated. The residue was purified by flash column chromatography with a mixture of Hexane/EtOAc (3/0.5-2/1) as eluent. Compound 24 was isolated as a brown solid (yields: 80%). ¹H NMR (400 MHz, CDCl₃): δ 1.49 (9H, s), 4.56 (2H, s), 6.77 (1H, d, ³J=2.0 Hz), 6.89 (1H, dd, ²J=8.0 Hz, ³J=2.0 Hz), 7.15 (1H, s), 7.33 (1H, d, ³J=8.0 Hz).

Phosphoramidate 1 (200 mg, 0.32 mmol) and Compound 24 (100 mg, 0.32 mmol) were suspended in THF (2.5 mL). CuSO₄ (80 mg, 0.32 mmol) and sodium ascorbate (63 mg, 0.32 mmol) were added followed by water (2.5 mL). The reaction was stirred in the dark for 18 h. After concentration, the aqueous phase was extracted twice with CH₂Cl₂ (25 mL). The combined organic layers were washed with brine, dried with MgSO₄, filtered and concentrated. Flash column chromatography of the residue hexane/AcOEt (2/1) furnished Compound 25 as a yellow solid (yields: 91%). ¹H NMR (400 MHz, CDCl₃): δ 0.86 (6H, t, ³J=6.7 Hz), 1.24 (44H, m), 1.49 (9H, s), 1.65 (4H, q, ³J=6.7 Hz), 1.97 (8H, m), 3.28 (1H, m), 3.99 (4H, m, ³J=6.7 Hz), 4.28 (2H, m), 4.60 (2H, s), 5.31 (4H, m), 6.84 (1H, d, ³J=2.0 Hz), 6.97 (1H, dd, ²J=8.0 Hz ³J=2.0 Hz), 7.56 (1H, d, ³J=8.0 Hz), 8.49 (1H, s), 8.55 (1H, s). ³¹P NMR (162 MHz, CDCl₃): 8.90 (s).

Phosphoramidate 25 (800 mg, 0.83 mmol) and TFA (1 mL) were suspended in dry CH₂Cl₂ (5 mL) and stirred at room temperature for 3.5 h. The crude was diluted with CH₂Cl₂ (5 mL), washed with water, brine and an aqueous 10% citrate solution. The organic layer was dried with MgSO₄, filtered and concentrated to furnish Compound 26 as an orange solid in quantitative yield. ¹H NMR (400 MHz, CDCl₃-CD₃OD): δ 0.82 (6H, t, ³J=6.7 Hz), 1.21 (44H, m), 1.61 (4H, m, ³J=6.7 Hz), 1.95 (8H, m), 3.94 (4H, m, ³J=6.7 Hz), 4.23 (2H, d, ³J=12.0 Hz), 4.66 (2H, s), 5.31 (4H, m), 6.87 (1H, d, ³J=2.0 Hz), 6.96 (1H, dd, ²J=8.0 Hz, ³J=2.0 Hz), 7.54 (1H, d, ³J=8.0 Hz), 8.44 (1H, s), 8.51 (1H, s). ³¹P NMR (162 MHz, CDCl₃-CD₃OD): 9.05 (s).

Phosphoramidate 26 (100 mg, 0.07 mmol) and mPEG-1900(OH) (141 mg, 0.11 mmol) were suspended in dry CH₂Cl₂ (5 mL). At room temperature, DMAP (4.7 mg, 0.04 mmol) then EDCI (15 mg, 0.11 mmol) were added and the mixture was stirred for 2 days under nitrogen atmosphere. The crude material was diluted with CH₂Cl₂ (10 mL), washed with water and brine, dried with CH₂Cl₂/MeOH (95/5). Compound 27a was obtained as a colourless solid (yields: 92%). ¹H NMR (400 MHz, CDCl₃): δ 0.82 (6H, t, ³J=6.7 Hz), 1.21 (44H, m), 1.61 (4H, m, ³J=6.7 Hz), 1.95 (8H, m), 3.34 (3H, s), 3.41-3.79 (H, m), 3.99 (4H, m, ³J=6.7 Hz), 4.27 (2H, m), 4.36 (2H, m), 4.75 (2H, s), 5.31 (4H, m), 6.87 (1H, d, ³J=2.0 Hz), 6.96 (1H, dd, ²J=8.0 Hz, ³J=2.0 Hz), 7.54 (1H, d, ³J=8.0 Hz), 8.44 (1H, s), 8.52 (1H, s). ³¹P NMR (162 MHz, CDCl₃): 8.84 (s).

Phosphoramidate 26 (100 mg, 0.07 mmol) and mPEG-1900(NH₂) (141 mg, 0.11 mmol) were suspended in dry CH₂Cl₂ (5 mL). At room temperature, DMAP (4.7 mg, 0.04 mmol) then EDCI (15 mg, 0.11 mmol) were added and the mixture was stirred for 2 days under nitrogen atmosphere. The crude material was diluted with CH₂Cl₂ (10 mL), washed with water and brine, dried with MgSO₄, filtered and concentrated to furnish an orange oil which was purified by column chromatography with CH₂Cl₂/MeOH (95/5) to produce Compound 27b as a white solid (yields: 55%). ¹H NMR (400 MHz, CDCl₃): δ 0.82 (6H, t, ³J=6.7 Hz), 1.21 (44H, m), 1.61 (4H, m, ³J=6.7 Hz), 1.95 (8H, m), 3.34 (3H, s), 3.41-3.79 (H, m), 3.99 (4H, m, ³J=6.7 Hz), 4.27 (2H, m), 4.36 (2H, m), 4.75 (2H, s), 5.31 (4H, m), 6.87 (1H, d, ³J=2.0 Hz), 6.96 (1H, dd, ²J=8.0 Hz, ³J=2.0 Hz), 7.54 (1H, d, ³J=8.0 Hz), 8.44 (1H, s), 8.52 (1H, s). ³¹P NMR (162 MHz, CDCl₃): 8.84 (s).

### Synthesis of Compounds 48 and 49—Scheme 12

Compound 48 was synthesised following a procedure reported in P. Kele, X. Li, M. Link, K. Nagy, A. Herner, K. Lorincz, S. Beni, O. S. Wolfbeis, Org. Biomol. Chem. 2009, 7, 3486-3490, incorporated herein by reference in its entirety.

Phosphoramide 13 (55 mg, 0.07 mmol) and Compound 48 (50 mg, 0.07 mmol) were combined in a (2/1) mixture of DMF (2.5 mL) and water. CuSO₄.5H₂O (17 mg, 0.07 mmol) and sodium ascorbate (13.5 mg, 0.07 mmol) were added and the mixture was stirred in the dark for 48 h. The crude mixture was diluted with 5 mL of water and extracted several times with CH₂Cl₂. The organic layers were combined, washed with water, dried with MgSO₄, filtered and concentrated. The residue was purified by column chromatography CH₂Cl₂/MeOH (9/1-7/1 v/v) to give Compound 49 as an dark blue oil (yield 295 mg (32%)). ¹H NMR (400 MHz, DMSO-d₆/CDCl₃): δ=0.82 (t, J=6.7 Hz, 6H, CH₃), 1.20 (m, 44H, CH₂), 1.40 (s, 12H, CH₃), 1.51 (m, 8H, CH₂), 1.72 (q, 4H, CH₂), 1.94 (m, 8H, CH₂), 2.72 (m, 4H, CH₂), 2.82 (m, 2H, CH₂), 3.34 (s, 4H, CH₂), 3.43-3.55 (m, 12H, CH₂-TEG), 3.79 (m, 4H, CH₂), 3.89 (m, 4H, CH₂OP), 4.52 (m, 2H, CH₂), 4.68 (m, 1H, NH), 4.96 (m, 2H, CH₂), 5.26 (m, 4H, CH═CH), 5.52 (d, J=12.4 Hz, 2H, CH₂), 6.98 (m, 2H, ═CH), 7.08 (m, 2H, ═CH), 7.22 (m, 2H, ═CH), 7.30 (m, 2H, ═CH), 7.75 (m, 2H, ═CH), 8.13 (s, 1H, H-triazol). ¹³C NMR (125 MHz, DMSO-d₆/CDCl₃): 14.4, 22.1, 25.1, 26.6, 28.0-31.4, 32.0, 40.3, 47.1, 50.1, 53.0, 66.1, 66.2-72.8, 76.9, 96.3, 109.0, 122.5, 123.5, 126.5, 128.2, 129.2-133.1, 134.4, 139.9, 140.9, 143.90, 165.9, 167.7. ³¹P NMR (162 MHz, CDCl₃): 9.8. HRMS (MALDI): m/z calcd. for C₈₄H₁₃₆N₇O₁₂PS₂ [(M+K)⁺]: 1568.906. Found: 1569.020. (λ_{max,exc}=670 nm (EtOH); λ_{max,em}=713 nm (EtOH); εₘₐₓ 190000).

### Example 3

### Targeting Fluorescent Phospholipids—Precursors and Functionalizable Phospholipids

### Synthesis of Compounds 28, 29, 30 and 31

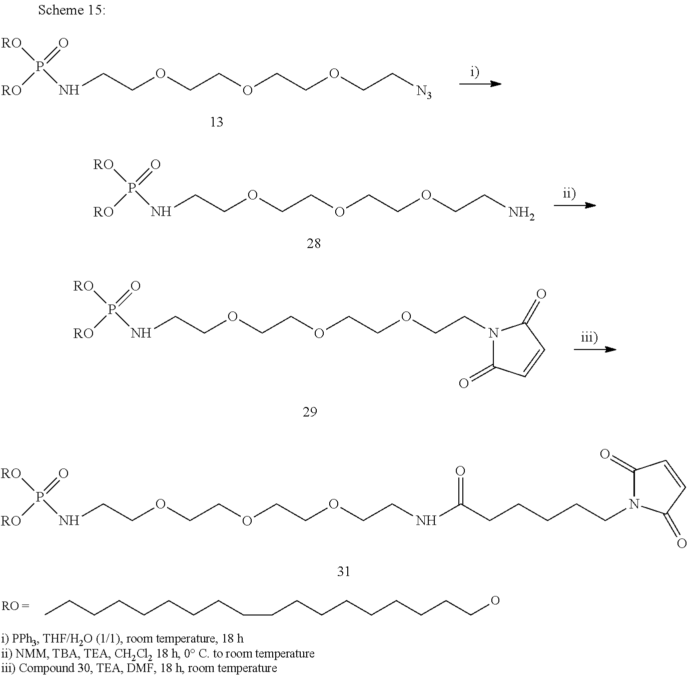

Phosphoramidate 13 (500 mg, 0.63 mmol) was dissolved in a mixture of THF/H₂O (1/1) and PPh₃ (207 mg, 0.80 mmol) was added portionwise. The reaction was stirred at room temperature for 18 h. The crude was concentrated under reduced pressure and column chromatography with CH₂Cl₂/MeOH/NH₄OH (28/2/0.1) gave Compound 28 as a colourless oil (yields: 57%). ¹H NMR (400 MHz, CDCl₃): δ 0.86 (6H, t, J=6.7 Hz), 1.26 (44H, m), 1.65 (4H, m), 1.99 (8H, m), 2.87 (2H, t, ³J=5.2 Hz), 3.06 (2H, m), 3.52 (4H, t, ³J=5.0 Hz), 3.63 (8H, m), 3.96 (4H, m, ³J=6.0 Hz), 5.28 (4H, m).

At 0° C., phosphoramidate 28 (250 mg, 0.36 mmol), NMM (100 mg, 0.36 mmol) and TBA (110 mg, 0.36 mmol) were suspended in dry CH₂Cl₂ (5 mL) under N₂ atmosphere with stirring. TEA (232 mg, 0.36 mmol) was added in 5 min and the reaction was kept at 0° C. for 10 more minutes. The mixture was allowed to warm to room temperature, then aqueous saturated NaHCO₃ (8 mL) was added under vigorous stirring for 18 h. The organic layer was separated, dried with MgSO₄, filtered and concentrated. Column chromatography with CH₂Cl₂/EtOH (30/1-30/1.5) furnished the Compound 29 as a colourless oil (yields: 45%). ¹H NMR (400 MHz, CDCl₃): δ 0.86 (6H, t, J=6.7 Hz), 1.26 (44H, m), 1.65 (4H, q, ³J=6.7 Hz), 1.99 (8H, m), 3.12 (1H, m), 3.48 (2H, t, ³J=5.0 Hz), 3.58-3.65 (10H, m), 3.70 (2H, t, ³J=5.0 Hz), 3.96 (4H, m, ³J=6.7 Hz), 5.33 (4H, m), 6.71 (2H, s).

5-maleimideocaproic acid (500 mg, 2.4 mmol), NHS (327 mg, 2.84 mmol) and EDCI (544 mg, 2.84 mmol) were suspended in dry DMF (5 mL) under N₂ atmosphere. The reaction was stirred at room temperature overnight. The solvent was discarded and the residue was dissolved in CH₂Cl₂, washed with water and brine, then dried with MgSO₄, filtered and concentrated. The waxy product, Compound 30, was used without further purification. ¹H NMR (400 MHz, CDCl₃): δ 1.38 (2H, m), 1.58 (2H, m), 1.65 (2H, m), 2.57 (2H, m, ³J=7.0 Hz), 2.80-2.93 (4H, m), 3.60 (2H, t, ³J=7.0 Hz), 6.66 (2H, s).

Phosphoramidate 28 (500 mg, 0.26 mmol), Compound 30 (88 mg, 0.27 mmol) and TEA (88 mg, 0.27 mmol) were suspended in dry DMF (5 mL) under N₂ atmosphere and stirred at room temperature overnight. The reaction product was concentrated and column chromatography of the residual oil CH₂Cl₂/EtOH (30/1.2-30/1.5) furnished the Compound 31 as a colourless oil (yields: 45%). ¹H NMR (400 MHz, CDCl₃): δ 0.86 (6H, t, ³J=6.7 Hz), 1.26 (44H, m), 1.63-1.71 (8H, m), 1.65 (4H, q, ³J=6.7 Hz), 1.99 (8H, m), 2.17 (2H, t, ³J=7.6 Hz), 3.06 (2H, m), 3.41 (1H, m), 3.43-3.70 (14H, m), 3.96 (4H, m, J=6.7 Hz), 5.33 (4H, m), 6.55 (1H, bs), 6.72 (2H, s).

### Example 4

### Targeting Fluorescent Phospholipids—Targeting Fluorescent Phospholipids

### Synthesis of Compounds 32, 33, 34 and 35

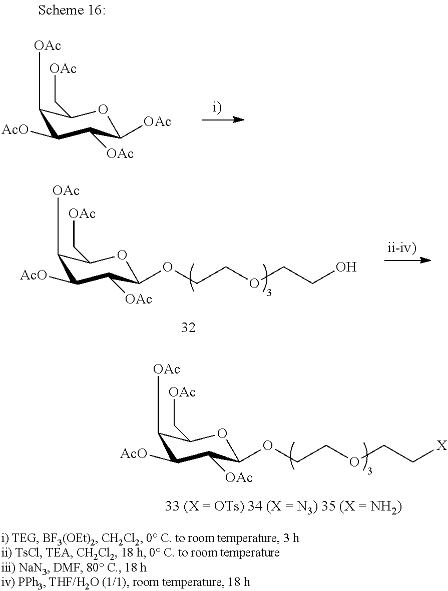

β-D-Galactopyranose pentaacetate (5.0 g, 12.8 mmol) and TEG (1.91 g, 15.3 mmol) were mixed in dry CH₂Cl₂ (35 ml) and stirred for 1 h with molecular sieves. Then, the mixture was cooled to 0° C., boron trifluoride etherate (18.5 g, 130 mmol) was added and stiffing continued for 3 h at room temperature. The mixture was diluted with CH₂Cl₂ (100 ml), washed with water, saturated aqueous NaHCO₃ and brine, dried over MgSO₄ and concentrated. Column chromatography of the residual oil (AcOEt/EtOH 28/2) produced Compound 32 as a colourless oil (yields: 60%). ¹H NMR (400 MHz, CDCl₃): δ 1.96 (s, 3H), 2.03 (s, 3H), 2.04 (s, 3H), 2.13 (s, 3H), 2.60 (bs, 1H), 3.58-3.74 (m, 15H), 3.92 (dt, J=7.0 Hz, J=10.0 Hz, 1H), 4.13 (dd, J=7.0 Hz, J=10.0 Hz, 1H), 4.55 (d, J=8.0 Hz, 1H), 4.99 (1H, dd, J=4.0 Hz, J=10.0 Hz, 1H), 5.18 (dd, J=8.0 Hz, J=10.0 Hz, 1H), 5.37 (1H, dd, J=1.0 Hz, J=4.0 Hz, 1H).

Compound 32 (2.8 g, 5.34 mmol) and TsCl (1.53 g, 8.0 mmol) in dry CH₂Cl₂ (80 ml) were cooled to 0° C. and TEA (973 mg, 9.61 mmol) was added. The mixture was stirred at room temperature for 20 h. Water was added and the organic layer washed with water and brine, dried with MgSO₄ and concentrated. Column chromatography of the residual oil (AcOEt:hexane 3/1) produced Compound 33 as a colourless oil (yields: 83%).

Compound 33 (3 g, 4.42 mmol) and sodium azide (575 mg, 8.84 mmol) in dry DMF (50 ml) were stirred at 80° C. for 18 h. The solvent was evaporated under reduced pressure and the crude was diluted with AcOEt, washed with water and saturated NH₄Cl, dried over MgSO₄ and concentrated. Flash column chromatography of the residual oil (AcOEt:hexane 1/1) produced Compound 34 as colourless oil (yields: 80%). ¹H NMR (400 MHz, CDCl₃): δ 1.86 (s, 3H), 2.01 (s, 3H), 2.07 (s, 3H), 2.11 (s, 3H), 3.30 (t, J=5.0 Hz, 2H), 3.56-3.64 (m, 15H), 3.82 (dt, J=7.0 Hz, J=10.0 Hz, 1H), 4.08 (dd, J=7.0 Hz, J=10.0 Hz, 1H), 4.52 (d, J=8.0 Hz, 1H), 4.95 (1H, dd, J=4.0 Hz, J=10.0 Hz, 1H), 5.13 (dd, J=8.0 Hz, J=10.0 Hz, 1H), 5.32 (1H, dd, J=1.0 Hz, J=4.0 Hz, 1H).

To Compound 34 (1.7 g, 3.15 mmol) and PPh₃ (1.24 g, 4.30 mmol) in THF (100 ml) was added water (30 mL) and the mixture was stirred at room temperature for 2 days. The solvent was evaporated under reduced pressure and the crude was purified by column chromatography (AcOEt/EtOH/NH₄OH 2/1/10%) produced Compound 35 as colourless oil. ¹H NMR (400 MHz, CDCl₃): δ 1.86 (s, 3H), 2.01 (s, 3H), 2.07 (s, 3H), 2.11 (s, 3H), 3.35 (m, J=4.0 Hz, 2H), 3.56-3.64 (m, 15H), 3.82 (dt, J=7.0 Hz, J=10.0 Hz, 1H), 4.08 (dd, J=7.0 Hz, J=10.0 Hz, 1H), 4.52 (d, J=8.0 Hz, 1H), 4.95 (1H, dd, J=4.0 Hz, J=10.0 Hz, 1H), 5.12 (dd, J=8.0 Hz, J=10.0 Hz, 1H), 5.31 (1H, dd, J=1.0 Hz, J=4.0 Hz, 1H).

### Synthesis of Compounds 37, 38 and 39

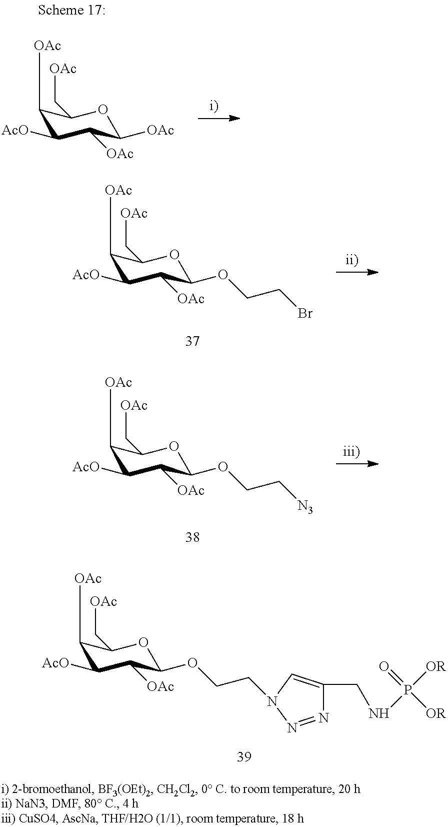

β-D-Galactopyranose pentaacetate (5.0 g, 12.8 mmol) and 2-bromoethanol (1.91 g, 15.3 mmol) were mixed in dry CH₂Cl₂ (50 ml) and stirred for 1 h with molecular sieves. Then; the mixture was cooled to 0° C., boron trifluoride etherate (9.0 mL) was added and stiffing continued for 20 h at room temperature under N₂ atmosphere. The crude was diluted with CH₂Cl₂ (20 ml) and water (50 mL) and the phases were separated. The organic layer was washed with saturated aqueous NaHCO₃ and brine, dried over MgSO₄ and concentrated. Column chromatography of the residual oil (AcOEt:hexane 1/1) produced Compound 37 as a colourless waxy solid (yields: 60%). ¹H NMR (400 MHz, CDCl₃): δ 1.90 (s, 3H), 2.02 (s, 3H), 2.05 (s, 3H), 2.10 (s, 3H), 3.42 (t, J=6.0 Hz, 2H), 3.58 (dt, J=10.0 Hz, J=6.0 Hz, 1H), 3.89 (dt, J=1.0 Hz, J=7.0 Hz, 1H), 4.13, 4.12 (dd, J=7.0 Hz, J=10.0 Hz, 1H), 4.51 (d, J=8.0 Hz, 1H), 4.99 (1H, dd, J=4.0 Hz, J=10.0 Hz, 1H), 5.18 (dd, J=8.0 Hz, J=10.0 Hz, 1H), 5.35 (1H, dd, J=1.0 Hz, J=4.0 Hz, 1H).

Compound 37 (3 g, mmol) and sodium azide (860 mg, 13.0 mmol) in dry DMF (50 ml) were stirred at 80° C. for 4 h under N₂ atmosphere. The solvent was evaporated under reduced pressure and the crude was diluted with AcOEt, washed with water and saturated NH₄Cl, dried over MgSO₄ and concentrated. Flash column chromatography of the residual oil (AcOEt:hexane 1/1) produced Compound 38 as colourless oil (yields: 90%). ¹H NMR (400 MHz, CDCl₃): δ 1.90 (s, 3H), 2.02 (s, 3H), 2.05 (s, 3H), 2.10 (s, 3H), 3.30 (t, J=6.0 Hz, 2H), 3.75-3.48 (dt, J=10.0 Hz, J=6.0 Hz, 1H), 3.89 (dt, J=1.0 Hz, J=7.0 Hz, 1H), 4.15 (dd, J=7.0 Hz, J=11.0 Hz, 1H), 4.13 (dd, J=7.0 Hz, J=11.0 Hz, 1H), 4.53 (d, J=8.0 Hz, 1H), 4.99 (dd, J=3.0 Hz, J=10.0 Hz, 1H), 5.20 (dd, J=10.0 Hz, J=8.0 Hz, 1H), 5.36 (dd, J=1.0 Hz, J=3.0 Hz, 1H)

General Procedure for Synthesis of Galactosylated Phosphoramidates According to an Embodiment—Scheme 17

Phosphoramidate 2 (100 mg, 0.16 mmol) and Compound 38 (67 mg, 0.16 mmol) were combined in THF (2.5 mL). CuSO₄ (40 mg, 0.16 mmol) and sodium ascorbate (32 mg, 0.16 mmol) were added along with water (2.5 mL) and the reaction was stirred in the dark overnight. THF was evaporated under reduced pressure and the aqueous layer was extracted twice with CH₂Cl₂ (20 ml). The combined organic layers were washed with brine, dried with MgSO₄, filtered and concentrated. Column chromatography of the residue produced Compound 39 (AcOEt/MeOH 5/1) (yields: 75%). ¹H NMR (400 MHz, CDCl₃): δ 0.88 (t, J=6.7 Hz, 6H), 1.26 (m, 28H), 1.66 (m, J=6.7 Hz, 8H), 1.90 (s, 3H), 2.02 (s, 3H), 2.05 (s, 3H), 2.17 (s, 3H), 2.01 (m, 8H), 2.76 (m, 4H), 3.48 (bs, 1H), 3.88 (m, 2H, m), 3.99 (q, 4H), 4.18 (m, 2H), 4.21 (m, 2H), 4.42 (d, J=6.7 Hz, 1H), 4.50 (m, 1H), 4.58 (m, 2H), 4.94 (dd, J=6.7 Hz, 1H), 5.12 (m, 1H), 5.37 (m, 8H), 5.39 (m, 1H), 7.56 (s, 1H). ³¹P NMR (162 MHz, CDCl₃): 8.48 (s). ¹³C NMR (300 MHz, CDCl₃): δ 14.41, 20.75, 22.57-31.90, 37.10, 49.10, 61.18, 66.18, 66.72, 66.81, 67.81), 70.53, 70.92, 100.77, 123.32, 127.90-130.22, 169.62-170.37. ¹³C NMR (300 MHz, CDCl₃): 14.45, 23.02, 25.89, 27.55-32.94, 67.01, 71.65, 76.00, 130.12-130.32. HRMS (MALDI): m/z calc. for C₃₉H₇₄NO₃P [M+Na]: 658.53. Found: 658.453

### Synthesis of Compounds 50 and 51

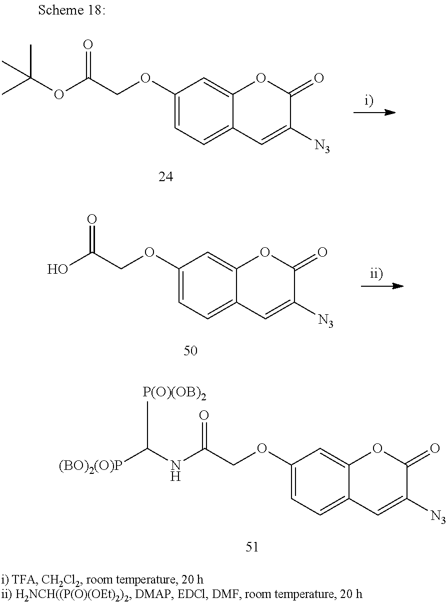

Compound 24 (900 mg, 0.50 mmol) was dissolved in dry CH₂Cl₂ (20 ml), TFA (3 mL) was added and the reaction was stirred at room temperature for 20 h. The crude was diluted with CH₂Cl₂ (50 ml), washed with water and a 10% aqueous citrate solution, dried with MgSO₄ and concentrated. Compound 50, as an orange solid, was used without further purification (yields: 65%). ¹H NMR (400 MHz, d₆-DMSO): δ 4.80 (2H, s), 6.96 (dd, 1H, ²J=8.0 Hz, ³J=2.0 Hz), 7.02 (d, 1H, ³J=2.0 Hz), 7.55 (d, 1H, ³J=8.0 Hz), 7.62 (s, 1H).

Compound 50 (200 mg, 0.77 mmol) was dissolved in dry DMF (20 ml), aminomethylene bisphosphonate (279 mg, 0.92 mmol), DMAP (112 mg, 0.77 mmol) and EDCI (176 mg, 0.92 mmol) were added and the reaction was stirred at room temperature for 20 h. The solvent was evaporated and the residue was suspended in CHCl₃ (20 ml). The organic layer was washed with water and brine, dried with MgSO₄ and concentrated. Column chromatography CH₂Cl₂/MeOH (30/1) gave Compound 51 as a colourless oil. ¹H NMR (400 MHz, d₆-DMSO): δ 1.41 (m, 12H), 4.30 (m, 8H), 4.72 (s, 2H), 6.95 (d, 1H, ³J=2.0 Hz), 7.55 (dd, 1H, ²J=8.0 Hz, ³J=2.0 Hz), 7.36 (s, 1H), 7.57 (d, 1H, ³J=8.0 Hz).

1. Lipophilic compound having the general formula (I): 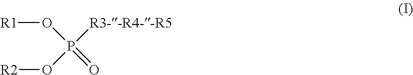
wherein:
(a) R1 and R2 are each independently a linear or branched, saturated or unsaturated C2-C24 alkyl, or a linear or branched, saturated or unsaturated C2-C24 monoalkenyl or polyalkenyl, the polyalkenyl having from 2 to 4 double bonds, or a linear or branched, saturated or unsaturated C2-C24 monoalkinyl or polyalkinyl, the polyalkinyl having from 2 to 4 triple bonds;
(b) R3 is selected from O, S, —C(R6)₂—, —CH(R7)-, —C(S)—N(R6)-, —CH(SR7)-S—S— or —N(R6)- wherein R6 is a hydrogen atom or a C1-C4 alkyl, and R7 is a C1-C4 alkyl;
(c) R4 comprises:
(c1) at least one junction function selected from an ether group, a thioether group, an ester group, an amide group, a thioamide group, a carbonyl group, a carbamate group, an urea group, a thiourea group, a disulfide group and a 1,2,3-triazole; or
(c2) at least one linker comprising a linear or branched, saturated or unsaturated hydrocarbon chain, the hydrocarbon chain being unsubstituted or substituted by one or a plurality of heteroatoms selected from N, O or S, interrupted and/or terminated by one or a plurality of junction functions selected from an ether group, a thioether group, an ester group, an amide group, a thioamide group, a carbonyl group, a carbamate group, an urea group, a thiourea group, a disulfide group and a 1,2,3-triazole, and optionally interrupted and/or terminated by one or a plurality of groups selected from C1-C4 alkyl radicals, C1-C4 alkoxy radicals and aryl radicals; and
(d) R5 is at least one chemical fluorescent group or at least one targeting group.

2. The compound according to claim 1, wherein R5 is at least one chemical fluorescent group and wherein the lipohilic compound has the general formula (II): 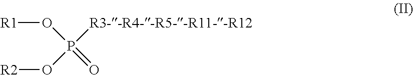
wherein:
(e) R11 comprises:
(e1) at least one junction function selected from an ether group, a thioether group, an ester group, an amide group, a thioamide group, a carbonyl group, a carbamate group, an urea group, a thiourea group, a disulfide group and a 1,2,3-triazole; or
(e2) at least one linker comprising a linear or branched, saturated or unsaturated hydrocarbon chain, the hydrocarbon chain being unsubstituted or substituted by one or a plurality of heteroatoms selected from N, O or S, interrupted and/or terminated by one or a plurality of junction functions selected from an ether group, a thioether group, an ester group, an amide group, a thioamide group, a carbonyl group, a carbamate group, an urea group, a thiourea group, a disulfide group and a 1,2,3-triazole, and optionally interrupted and/or terminated by one or a plurality of groups selected from C1-C4 alkyl radicals, C1-C4 alkoxy radicals and aryl radicals; and
(f) R12 is at least one targeting group.

3. The compound of claim 2, wherein, when R11 comprises a linker, said linker comprises a chain terminated by two junction functions, a first junction function being covalently bonded to R5 and a second junction function being covalently bonded to R12.

4. The compound of claim 1, wherein R1 and R2 are each independently a linear or branched, saturated or unsaturated C10-C24 alkyl, a linear or branched, saturated or unsaturated C10-C24 monoalkenyl or a linear or branched, saturated or unsaturated C10-C24 monoalkinyl.

5. The compound of claim 1, wherein R3 is —N(H)—.

6. The compound of claim 1, wherein the linker is a C1-C10.

7. The compound of claim 1, wherein the linker is a C1-C60 polyethyleneglycol.

8. The compound of claim 7, wherein the linker is a tetraethyleneglycol.

9. The compound of claim 1, wherein the junction function is one selected from the group consisting of an amide group, a 1,2,3-triazole, a thioether group, a thioamide group, an urea group or a thiourea group.

10. The compound of claim 1, wherein the at least one targeting group is selected from saccharides, disaccharides, polysaccharides, anisamides, derivatives for sigma receptor targeting, peptides, folate, antibody, phosphonic acid, di-phosphonic acid, or polyethyleneglycol.

11. The compound of claim 1, wherein, when R4 comprises a linker, said linker comprises a chain terminated by two junction functions, a first junction function being covalently bonded to R3 and a second junction function being covalently bonded to R5.

12. A method for the preparation of a lipophilic compound according to claim 1, the method comprising:
at least one coupling step between a Compound (A) and a Compound (B) by the formation of at least one covalent bond of the C-Het type or of the Het-Het′ type, wherein Het and Het′ are the same or different heteroatom(s), wherein Compound (A) is a compound of general formula (III): 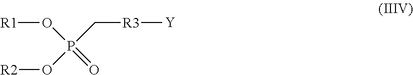
wherein Y is a grafting terminal functional group or unit selected from alcohol, phenol, thiol, amine, azide, dithioester, acylchloride, carboxylic acid, maleimide, isocyanates, isothiocyanate, alkyne, alkene, aziridine, epoxide, or vinyl;
wherein Compound (B) comprises R5 and a grafting terminal functional group or unit Z selected from alcohol, phenol, thiol, amine, azide, dithioester, acylchloride, carboxylic acid, maleimide, isocyanates, isothiocyanate, alkyne, alkene, aziridine, epoxide, or vinyl; and
wherein Z is capable of reacting with Y to form said covalent bond between Compound (A) and Compound (B).

13. The method of claim 12, wherein Compound (A) and Compound (B) are coupled through a Compound (C) by the formation of at least one covalent bond of the C-Het type or of the Het-Het′ type, wherein Het and Het′ are the same or different heteroatom(s), wherein Compound (C) comprises R4, R4 comprising at least one linker, and two grafting terminal functional groups or units W and X selected each independently from alcohol, phenol, thiol, amine, azide, dithioester, acylchloride, carboxylic acid, maleimide, isocyanates, isothiocyanate, alkyne, alkene, aziridine, epoxide, or vinyl, and
wherein Z is capable of reacting with X to form a covalent bond between Compound (B) and Compound (C) and Y is capable of reacting with W to form a covalent bond between Compound (A) and Compound (C).

14. The method of claim 12, further comprising:
at least one further coupling step between Compound (B) and a Compound (D) by the formation of at least one covalent bond of the C-Het type or of the Het-Het′ type, wherein Het and Het′ are the same or different heteroatom(s), wherein R5 of Compound (B) is a chemical fluorescent group, wherein Compound (B) further comprises a grafting terminal functional group or unit U selected from alcool, phenol, thiol, amine, azide, dithioester, acylchloride, carboxylic acid, maleimide, isocyanates, isothiocyanate, alkyne, alkene, aziridine, epoxide, or vinyl,
wherein Compound (D) comprises R12 and a grafting terminal functional group or unit M selected from alcool, phenol, thiol, amine, azide, dithioester, acylchloride, carboxylic acid, maleimide, isocyanates, isothiocyanate, alkyne, alkene, aziridine, epoxide, or vinyl,
wherein M is capable of reacting with U to form said covalent bond between Compound (B) and Compound (D).

15. The method of claim 14, wherein Compound (B) and Compound (D) are coupled through a Compound (E) by the formation of at least one covalent bond of the C-Het type or of the Het-Het′ type, wherein Het and Het′ are the same or different heteroatom(s), wherein Compound (E) comprises R11, R11 comprising a linker, and two grafting terminal functional groups or units T and Q selected each independently from alcool, phenol, thiol, amine, azide, dithioester, acylchloride, carboxylic acid, maleimide, isocyanates, isothiocyanate, alkyne, alkene, aziridine, epoxide, or vinyl,
wherein M is capable of reacting with Q to form a covalent bond between Compound (D) and Compound (E) and T is capable of reacting with U to form a covalent bond between Compound (B) and Compound (E).
